(19) United States
(12) Patent Application Publication
(10) Pub. No.: US 2025/0283711 A1
(43) Pub. Date: Sep. 11, 2025

(54) SYSTEMS AND METHODS FOR MULTIPATH IMAGING

(71) Applicant: Provincial Health Services Authority, Vancouver (CA)

(72) Inventors: Jeanie MALONE, Vancouver (CA); Calum MACAULAY, Vancouver (CA); Adrian TANSKANEN, Burnaby (CA); Pierre LANE, Vancouver (CA)

(21) Appl. No.: 19/214,633

(22) Filed: May 21, 2025

Related U.S. Application Data
(63) Continuation of application No. PCT/CA2024/050069, filed on Jan. 22, 2024.
(60) Provisional application No. 63/440,954, filed on Jan. 25, 2023.

(51) Int. Cl. G01B 9/02091; A61B 5/00

(52) U.S. Cl. CPC G01B 9/02091; A61B 5/0066; A61B 5/0084; A61B 2576/00

(57) ABSTRACT

A fiber optic imaging system generates multipath image data. The system comprises: an optical coherence tomography (OCT) system comprising an OCT fiber optically connected to an interferometric detector; a multi-clad fiber (MCF) for receiving sampled light that has interacted with a sample and propagating the sampled light as a fundamental MCF mode and one or more higher order MCF modes; and an optical joint for coupling the sampled light from the MCF into the OCT fiber, the optical joint configured to couple at least some light energy from both the fundamental MCF mode and the one or more higher order MCF modes into a fundamental mode of the OCT fiber where the at least some light energy propagates as OCT return light.

### CROSS-REFERENCE TO RELATED APPLICATIONS

This application is a continuation of Patent Cooperation Treaty (PCT) application No. PCT/CA2024/050069 entitled SYSTEMS AND METHODS FOR MULTIPATH IMAGING and having an international filing date of 22 Jan. 2024 which in turn claims priority from, and for the purposes of the United States of America the benefit under 35 U.S.C. § 119 of, U.S. application No. 63/440,954 filed 25 Jan. 2023 and entitled NOVEL METHODS FOR MULTIPATH CONTRAST IMAGING. All of the applications referred to in this paragraph are hereby incorporated herein by reference for all purposes.

### FIELD

This invention relates to fiber optic based optical imaging, such as endoscopy, for example. Particular embodiments provide methods and systems for optical coherence tomography (OCT) imaging using multi-clad fibers and corresponding imaging techniques.

### BACKGROUND

Optical endoscopes exploit the near-lossless transmission, narrow imaging cores and flexibility of fiber optics to provide high resolution, minimally invasive imaging in, for example, luminal organs deep within the body.

Low signal modalities such as contrast-free fluorescence imaging necessitate multi-mode fibers (MMF). MMF have high collection efficiencies from refractive index profiles (RIPs) with wide diameter and high numerical aperture (NA) cores.

Confocal endomicroscopy makes use of single-mode fibers (SMF) in place of a pinhole to maintain optical sectioning properties of a free-space system. Time-gated techniques such as OCT also require SMF to limit modal dispersion as additional modes introduce ghost images which can obscure the true image.

Optical fibers with more complex RIPs further enable more elaborate imaging schemes. For example, multi-type imaging comprising OCT and an additional imaging type makes use of double-clad fiber (DCF) which has a single-mode core for OCT, and a multimode inner-cladding for the additional imaging type. OCT provides volumetric imaging of subsurface tissue morphology; thus, functional imaging modalities which provide information regarding tissue biochemistry, such as autofluorescence imaging (AFI), may be used, for example, as the additional imaging type.

Utilizing complex RIP geometries to implement multi-type imaging in a single fiber (e.g. a DCF) typically has a cost. For instance, the doped cores of DCF generate additional background fluorescence when compared to a pure-silica MMF core, reducing fluorescence image quality. In the OCT type, imaging with DCF as opposed to SMF results in time shifted multipath artifacts, i.e., ghost images smeared in the A-line direction that may be superimposed on the fundamental image.

The distance (in the A-line direction) between the true image and any multipath artifacts is typically linearly related to the length of DCF used in the sample arm of the endoscopic system. In theory, the impacts of multipath artifacts could be significantly mitigated by using a long length of DCF (>5 m) in the sample arm. In practice, the length of DCF in the sample arm of an endoscopic imaging catheter is typically constrained to <2 m by mechanical and clinical needs; thus, in prior art techniques, these multipath artifact impacts are generally mitigated by folding the multipath artifacts over the zero-reference delay to prevent or mitigate superposition of the multipath artifacts over the fundamental image.

There is a general desire to provide systems and methods for OCT imaging that are capable of using a MCF (e.g. DCF) in the sample arm (e.g. to support other imaging modalities), while mitigating the effect of, or making use of, multipath artifacts.

There is a general desire for improved techniques for fiber optic based optical imaging.

### SUMMARY

One aspect of the invention provides a fiber optic imaging system for generating multipath image data. The system comprises: an optical coherence tomography (OCT) system comprising an OCT fiber optically connected to an interferometric detector; a multi-clad fiber (MCF) for receiving sampled light that has interacted with a sample and propagating the sampled light as a fundamental MCF mode and one or more higher order MCF modes; an optical joint for coupling the sampled light from the MCF into the OCT fiber, the optical joint configured to couple at least some light energy from both the fundamental MCF mode and the one or more higher order MCF modes into a fundamental mode of the OCT fiber where the at least some light energy propagates as OCT return light.

The imaging system may comprise a controller connected to the interferometric detector to receive, from the interferometric detector, OCT image data based on the OCT return light. The controller may be configured to: extract fundamental mode image data and higher order mode image data from the OCT image data, wherein the extraction is based on A-line coordinates of the OCT image data; and generate multipath image data (e.g. diagnostic image data) based on both of the fundamental mode image data and the higher order mode image data.

The controller may be configured to: extract the fundamental mode image data based on light energy from the fundamental MCF mode that is coupled into the fundamental mode of the OCT fiber; and extract the higher order mode image data based on light energy from the one or more higher order MCF modes that is coupled into the fundamental mode of the OCT fiber.

The controller may be configured to extract the fundamental mode image data and the higher order mode image data from the OCT image data using upper and lower A-line coordinate thresholds.

The MCF may introduce a phase delay between the fundamental MCF mode and the one or more higher order MCF modes.

The controller may be configured to extract the fundamental mode image data and the higher order mode image data based on the phase delay between the fundamental MCF mode and the one or more higher order MCF modes.

An amount of the phase delay may depend on a length of the MCF. The amount of the phase delay may be sufficient to enable extraction of the fundamental mode image data and the higher order mode image data from the OCT image data based on the A-line coordinates of the OCT image data.

The MCF may comprise a MCF core and at least one light-transmitting MCF cladding. The fundamental MCF mode may propagate substantially in the MCF core and the one or more higher order MCF modes may propagate substantially in the at least one MCF cladding.

The fundamental MCF mode may comprise light energy received from relatively low numerical aperture portions of the sampled light and the one or more higher order MCF modes may comprise light energy received from relatively high numerical aperture portions of the sampled light.

The MCF may propagate incident light from the optical joint toward the sample in an incident direction opposite to a direction of propagation of the sampled light. At least a portion of the incident light may interact with the sample to become the sampled light.

The incident light may comprise: fundamental mode incident light that propagates in the incident direction in a fundamental mode of the MCF; and higher order mode incident light that propagates in the incident direction in one or more higher order modes of the MCF.

The one or more higher order MCF modes of the sampled light may comprise a first component corresponding to the fundamental mode incident light that has interacted with the sample and a second component corresponding to the higher order mode incident light that has interacted with the sample.

The controller may be configured to extract the higher order mode image data based on light energy from the first component of the one or more higher order MCF modes of the sampled light that is coupled into the fundamental mode of the OCT fiber.

The controller may be configured to extract additional higher order mode image data from the OCT image data based on light energy from the second component of the one or more higher order MCF modes of the sampled light that is coupled into the fundamental mode of the OCT fiber.

The controller may be configured to extract the additional higher order mode image data based on A-line coordinates of the OCT image data.

The controller may be configured to extract the fundamental mode image data, the higher order mode image data and the additional higher order mode image data based on differential phase delay between different propagation modes introduced in the MCF.

The controller may be configured to generate multipath image data based at least in part on the additional higher order mode image data.

The optical joint may be configured to couple at least some light energy from both the fundamental MCF mode and the one or more higher order MCF modes into the fundamental of the OCT fiber by introducing an offset between the MCF and the OCT fiber.

The offset may comprise a spatial offset between an axis of the MCF and an axis of the OCT fiber. The offset may comprise an angular offset between an axis of the MCF and an axis of the OCT fiber.

The optical joint may comprise one or more optical components (e.g. lenses and/or mirrors) which introduce optical offset between the MCF and the OCT fiber.

The optical joint may comprise a fiber optic rotary joint (FORJ) configured to rotate the MCF about its axis relative to the sample.

The fundamental mode image data and the higher order mode image data may comprise volumetric data.

The controller may be configured to: process the fundamental mode image data to obtain two-dimensional processed fundamental mode image data; process the higher order mode image data to obtain two-dimensional processed higher order mode image data; and generate the multipath image data based on the two-dimensional processed fundamental mode image data and the two-dimensional processed higher order mode image data.

The controller may be configured to: process the fundamental mode image data to obtain the two-dimensional processed fundamental mode image data as a fundamental mode en face image by repeating: for each for each azimuthal coordinate (ϕ) and for each pullback axis coordinate in the fundamental mode image data, aggregating (e.g. adding or averaging) pixel values along the A-line direction between lower and upper fundamental mode A-line coordinate thresholds to obtain one corresponding pixel value of the two-dimensional processed fundamental mode image data; and process the higher order mode image data to obtain the two-dimensional processed higher order mode image data as a higher order mode en face image by repeating: for each for each azimuthal coordinate (ϕ) and for each pullback axis coordinate in the higher order mode image data, aggregating (e.g. adding or averaging) pixel values along the A-line direction between lower and upper higher order mode A-line coordinate thresholds to obtain one corresponding pixel value of the two-dimensional processed higher order mode image data.

The controller may be configured to generate the multipath image data based on a combination of the fundamental mode en face image and the higher order mode en face image.

The controller may be configured to generate the multipath image data based on a ratio of the fundamental mode en face image and the higher order mode en face image.

The MCF may comprise a double-cladded fiber (DCF).

The imaging system may comprise a second imaging type system, the second imaging type system configured to direct second incident light from the optical joint toward the sample by propagating the second incident light in the MCF, wherein at least a portion of the second incident light interacts with the sample to become second sampled light and wherein the second imaging type system is configured to collect the second sampled light which propagates back toward the optical joint in the MCF.

The imaging system may comprise a first light source that generates first incident light, at least a portion of which interacts with the sample to become the sampled light. The second imaging type system may comprise a second light source for generating the second incident light.

A wavelength of the first incident light may be different than a wavelength of the second incident light.

The optical joint may comprise a wavelength division multiplexer configured to combine the first incident light and the second incident light to generate combined incident light.

The optical joint may comprise a fiber optic rotary joint (FORJ) configured to rotate the MCF about its axis relative to the sample and for coupling the combined incident light into the MCF.

The optical joint may comprise a multi-clad fiber coupler (MCFC) configured to separate the sampled light from the second sampled light.

The second imaging type system may comprise an optical detector configured to detect at least a portion of the second sampled light. The controller may be configured to generate a second type image based on the detected portion of the second sampled light.

The second type image may comprise an en face image.

The second imaging type system may comprise an autofluorescence imaging (AFI) system.

Another aspect of the invention provides a method for generating multipath image data. The method comprises: providing a multi-clad fiber (MCF); receiving sampled light that has interacted with the sample and propagating the sampled light in the MCF as a fundamental MCF mode and one or more higher order MCF modes; coupling the sampled light from the MCF into an optical coherence tomography (OCT) fiber, wherein coupling the sampled light comprises coupling at least some light energy from both the fundamental MCF mode and the one or more higher order MCF modes into a fundamental mode of the OCT fiber, where the at least some light energy propagates as OCT return light; generating OCT image data based on the OCT return light; extracting fundamental mode image data and higher order mode image data from the OCT image data, wherein the extracting is based on A-line coordinates of the OCT image data; and generating the multipath image data based on both of the fundamental mode image data and the higher order mode image data.

The fundamental mode image data may be based on light energy from the fundamental MCF mode that is coupled into the fundamental mode of the OCT fiber; and the higher order mode image data may be based on light energy from the one or more higher order MCF modes that is coupled into the fundamental mode of the OCT fiber.

Extracting the fundamental mode image data and higher order mode image data from the OCT image data may comprise using upper and lower A-line thresholds.

The MCF may introduce a phase delay between the fundamental MCF mode and the one or more higher order MCF modes.

Extracting the fundamental mode image data and higher order mode image data from the OCT image data may be based on the phase delay between the MCF mode and the one or more higher order MCF modes.

An amount of the phase delay may depend on a length of the MCF. The amount of the phase delay may be sufficient to enable extraction of the fundamental mode image data and the higher order mode image data from the OCT image data based on the A-line coordinates of the OCT image data.

The MCF may comprise a MCF core and at least one light-transmitting MCF cladding. The fundamental MCF mode may propagate substantially in the MCF core and the one or more higher order MCF modes may propagate substantially in the at least one MCF cladding.

The fundamental MCF mode may comprise light energy received from relatively low numerical aperture portions of the sampled light and the one or more higher order MCF modes may comprise light energy received from relatively high numerical aperture portions of the sampled light.

The MCF may propagate incident light toward the sample in an incident direction opposite to a direction of propagation of the sampled light. At least a portion of the incident light my interact with the sample to become the sampled light.

The incident light may comprise: fundamental mode incident light that propagates in the incident direction in a fundamental mode of the MCF; and higher order mode incident light that propagates in the incident direction in one or more higher order modes of the MCF.

The one or more higher order MCF modes of the sampled light may comprise a first component corresponding to the fundamental mode incident light that has interacted with the sample and a second component corresponding to the higher order mode incident light that has interacted with the sample.

Extracting the higher order mode image data may be based on light energy from the first component of the one or more higher order MCF modes of the sampled light that is coupled into the fundamental mode of the OCT fiber.

The method may comprise extracting additional higher order mode image data from the OCT image data based on light energy from the second component of the one or more higher order MCF modes of the sampled light that is coupled into the fundamental mode of the OCT fiber.

The method may comprise extracting the additional higher order mode image data based on A-line coordinates of the OCT image data.

The method may comprise extracting the fundamental mode image data, the higher order mode image data and the additional higher order mode image data based on differential phase delay between different propagation modes introduced in the MCF.

Generating the multipath image data may be based at least in part on the additional higher order mode image data.

Coupling at least some light energy from both the fundamental MCF mode and the one or more higher order MCF modes into the fundamental of the OCT fiber may comprise providing an offset between the MCF and the OCT fiber.

The offset may comprise a spatial offset between an axis of the MCF and an axis of the OCT fiber.

The offset may comprise an angular offset between an axis of the MCF and an axis of the OCT fiber.

Providing the offset may comprise locating one or more optical components (e.g. lenses and/or mirrors) in an optical path between the MCF and the OCT fiber to thereby introduce optical offset between the MCF and the OCT fiber.

Coupling at least some light energy from both the fundamental MCF mode and the one or more higher order MCF modes into the fundamental of the OCT fiber may comprise locating a fiber optic rotary joint (FORJ) configured to rotate the MCF about its axis relative to the sample in an optical path between the MCF and the OCT fiber.

The fundamental mode image data and the higher order mode image data may comprise volumetric data.

The method may comprise: processing the fundamental mode image data to obtain two-dimensional processed fundamental mode image data; Processing the higher order mode image data to obtain two-dimensional processed higher order mode image data; and generating the multipath image data based on the two-dimensional processed fundamental mode image data and the two-dimensional processed higher order mode image data.

Processing the fundamental mode image data to obtain the two-dimensional processed fundamental mode image data may comprise processing the fundamental mode image data to obtain a fundamental mode en face image by repeating: for each for each azimuthal coordinate (ϕ) and for each pullback axis coordinate in the fundamental mode image data, aggregating (e.g. adding or averaging) pixel values along the A-line direction between lower and upper fundamental mode A-line coordinate thresholds to obtain one corresponding pixel value of the two-dimensional processed fundamental mode image data. Processing the higher order mode image data to obtain the two-dimensional processed higher order mode image data may comprise processing the higher order mode image data to obtain a higher order mode en face image by repeating: for each for each azimuthal coordinate (ϕ) and for each pullback axis coordinate in the higher order mode image data, aggregating (e.g. adding or averaging) pixel values along the A-line direction between lower and upper higher order mode A-line coordinate thresholds to obtain one corresponding pixel value of the two-dimensional processed higher order mode image data.

Generating the multipath image data may comprise generating the multipath image data based on a combination of the fundamental mode en face image and the higher order mode en face image.

Generating the multipath image data may comprise generating the multipath image data based on a ratio of the fundamental mode en face image and the higher order mode en face image.

The MCF may comprise a double-cladded fiber (DCF).

The method may comprise: providing a second imaging type system; propagating second incident light toward the sample by propagating the second incident light in the MCF; wherein at least a portion of the second incident light interacts with the sample to become second sampled light; and collecting the second sampled light which propagates back toward the second imaging type system in the MCF.

The method may comprise: generating first incident light by a first light source, at least a portion of which interacts with the sample to become the sampled light; and generating the second incident light by a second light source.

A wavelength of the first incident light may be different than a wavelength of the second incident light.

The method may comprise combining the first incident light and the second incident light to generate combined incident light.

The method may comprise coupling the combined incident light into the MCF by a fiber optic rotary joint (FORJ).

The method may comprise separating the sampled light from the second sampled light using a multi-clad fiber coupler (MCFC).

The method may comprise: detecting at least a portion of the second sampled light; and generating a second type image based on the detected portion of the second sampled light.

The second type image may comprise an en face image.

The second imaging type system may comprise an autofluorescence imaging (AFI) system.

Another aspect of the invention provides a fiber optic imaging system for generating multipath image data. The system comprises: an optical coherence tomography (OCT) system comprising an OCT fiber optically connected to an interferometric detector; a multi-clad fiber (MCF) for receiving sampled light that has interacted with a sample and propagating the sampled light as a fundamental MCF mode and one or more higher order MCF modes; an optical joint for coupling the sampled light from the MCF into the OCT fiber, the optical joint providing an offset between the MCF and the OCT fiber to couple at least some light energy from both the fundamental MCF mode and the one or more higher order MCF modes into a fundamental mode of the OCT fiber where the at least some light energy propagates as OCT return light.

The imaging system may comprise any of the features, combinations of features and/or sub-combinations of features discussed above or elsewhere herein.

Another aspect of the invention provides apparatus having any new and inventive feature, combination of features, or sub-combination of features described herein.

Another aspect of the invention provides methods having any new and inventive steps, acts, combination of steps and/or acts or sub-combination of steps and/or acts described herein.

It is emphasized that the invention relates to all combinations of the above features, even if these are recited in different claims.

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is a schematic block diagram showing an fiber optic imaging system comprising an optical coherence tomography (OCT) system and a multi-clad fiber (MCF) according to an example embodiment.

FIG. 2A is a schematic diagram showing an optical joint having a spatial offset between two fibers which may be used in the imaging system of FIG. 1 according to an example embodiment.

FIG. 2B is a schematic diagram showing an optical joint having an angular offset between two fibers which may be used in the imaging system of FIG. 1 according to an example embodiment.

FIG. 3 is a schematic block diagram showing a fiber optic imaging system comprising multiple imaging types (one of which is an OCT system) and a multimode fiber (e.g. multi-clad fiber (MCF)) according to another example embodiment. FIG. 3A is a schematic block diagram of an optical joint of the FIG. 3 imaging system according to a particular embodiment.

FIGS. 4A and 4B are plots showing geometric and refractive properties of single-mode fiber (SMF) and double-clad fiber (DCF) respectively.

FIG. 5A is an enlarged view of an end of the sample arm of the FIG. 3 imaging system according to an example embodiment.

FIG. 5B is a perspective view of a visualization of OCT image data depicting the coordinates used to describe OCT image data according to an example embodiment.

FIG. 6 is a flowchart of a method for generating multipath image data (e.g. diagnostic image data) using an imaging system having a sample arm comprising a MCF (e.g. one of the imaging systems of FIG. 1 or FIG. 3) according to an example embodiment.

FIGS. 7A-D are schematic diagrams showing propagation of incident light, sampled light and OCT return light in a fiber optic imaging system according to an example embodiment.

FIG. 8A is a plan view of a so-called longitudinal section (A-line axis by pullback axis) of OCT image data corresponding to one azimuthal value according to an example implementation.

FIG. 8B is a plan view of an en face projection of fundamental mode data of OCT image data according to an example implementation.

FIG. 8C is a plan view of an en face projection of higher order mode data of OCT image data according to an example implementation.

FIG. 8D is a plan view of an en face multipath contrast image according to an example embodiment.

FIGS. 9A-E (collectively, FIG. 9) are depictions of a set of multi-imaging types experimental data obtained from in vivo imaging of a human lung.

FIGS. 10A-E (collectively, FIG. 10) are depictions of another set of multi-imaging types experimental data obtained from ex vivo imaging of a fallopian tube specimen.

FIGS. 11A-D (collectively, FIG. 11) are depictions of another set experimental data obtained from an ex vivo fallopian tube specimen which shows how multiple components of higher order mode image data may be extracted from the OCT image data.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Aspects of the invention provides systems and methods for generating multipath image data (e.g. diagnostic endoscopic image data) based on fundamental mode data and higher order mode data extracted from OCT image data.

One aspect of the invention provides a fiber optic imaging system for generating multipath image data (e.g. diagnostic endoscopic image data). The system comprises: an optical coherence tomography (OCT) system having an OCT fiber optically connected to an interferometric detector; a multi-clad fiber (MCF) for receiving sampled light that has interacted with a sample and propagating the sampled light as a fundamental MCF mode and one or more higher order MCF modes; and an optical joint for coupling the sampled light from the MCF into the OCT fiber. The optical joint is configured to couple at least some light energy from both the fundamental MCF mode and the one or more higher order MCF modes into a fundamental mode of the OCT fiber where the at least some light energy propagates as OCT return light. The cross-coupling of light energy from one or more higher order MCF modes into the fundamental mode of the OCT fiber may be enabled by spatial and/or angular offset between the MCF and the OCT fiber. For example, such an offset may be between the optical axes of the MCF and the OCT fiber, where these optical axes represent the directions of light propagation.

The system may comprise a controller connected to the interferometric detector to receive, from the interferometric detector, OCT image data based on the OCT return light. The controller may be configured to: extract fundamental mode image data and higher order mode image data from the OCT image data, wherein the extraction is based on A-line coordinates of the OCT image data; and generate the multipath image data based on both of the fundamental mode image data and the higher order mode image data.

Another aspect of the invention provides a method for generating multipath image data (e.g. diagnostic image data) using the system(s) described herein. The method comprises: coupling the sampled light from a MCF into an optical coherence tomography (OCT) fiber, wherein coupling the sampled light comprises coupling at least some light energy from both the fundamental MCF mode and the one or more higher order MCF modes into a fundamental mode of the OCT fiber, where the at least some light energy propagates as OCT return light; generating OCT image data based on the OCT return light; extracting fundamental mode image data and higher order mode image data from the OCT image data, wherein the extracting is based on A-line coordinates of the OCT image data; and generating the multipath image data based on both of the fundamental mode image data and the higher order mode image data.

Another aspect of the invention provides achieving multi-type fiber optic imaging system (e.g. an endoscopic imaging system) comprising two or more imaging types, for example, OCT and AFI.

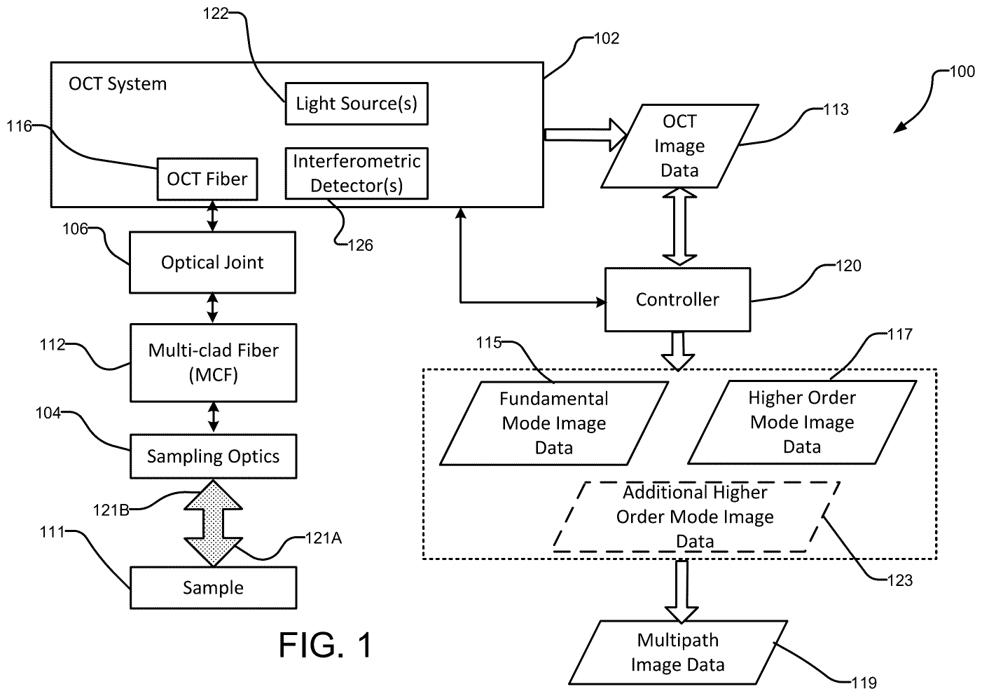

FIG. 1 is a schematic block diagram of a fiber optic (e.g. endoscopic) imaging system 100 according to an exemplary embodiment. System 100 comprises: an optical coherence tomography (OCT) system 102; sampling optics 104; a multi-clad fiber (MCF) 112; and an optical joint 106 for coupling light between MCF 112 and OCT system 102. System 100 may be used to generate multipath image data 119 from sample 111 (e.g. diagnostic image data from a body of a patient). For example, sampling optics 104 of system 100 may deliver incident light 121A to a sample 111 (e.g. the body of the patient) and MCF 112 may collect reflected/backscattered light (referred to herein as sampled light) 121B which has interacted with sample 111.

System 100 may comprise any suitable OCT system. System 100 of the illustrated embodiment comprises at least one light source(s) 122, typically a laser, and most typically a broadband source laser. In some embodiments, light source 122 comprises a swept-source laser. Light source 122 may emit light in any suitable wavelength range. Light as used herein should be understood to comprise electromagnetic radiation of any suitable wavelength. By way of non-limiting example, in some embodiments, light source 122 emits an infrared light. In a non-limiting example embodiment, light source 122 emits light in a wavelength range of about 1260 nm to about 1360 nm. System 100 of the illustrated embodiment also comprises an interferometric detector 126 for generating OCT image data 113 (which may be based on interferometric data (not explicitly shown) which is in turn based on interferometric optical path length differences) as is known to those skilled in the art. In some embodiments, interferometric data generated by interferometric detector 126 may be processed (e.g. by controller 120 and/or some other suitably configured processor (e.g. within OCT system 102)) to generate OCT image data 113.

System 100 further comprises an OCT fiber 116 which is used to couple incident light from light source 122 into sampling optics 104 via optical joint 106 and MCF 112. OCT fiber 116 may comprise any suitable optical fiber. In some embodiments, OCT fiber 116 comprises a single-mode fiber (SMF). In some embodiments, OCT fiber 116 comprises a double-clad fiber (DCF). In some embodiments, OCT fiber 116 comprises a multi-clad fiber (MCF). In some embodiments, OCT fiber 116 comprises a few mode fiber (FMF).

Incident light from light source 122 propagates through OCT fiber 116 and is coupled into MCF 112 by optical joint 106. In some embodiments, optical joint 106 comprises a fiber-optic rotary joint (FORJ). Any suitable FORJ may be used in optical joint 106. MCF 112 is capable of propagating light in multiple modes. Incident light originating from light source 122 propagates through MCF 112 before being directed by sampling optics 104 to impinge on sample 111 as incident light 121A.

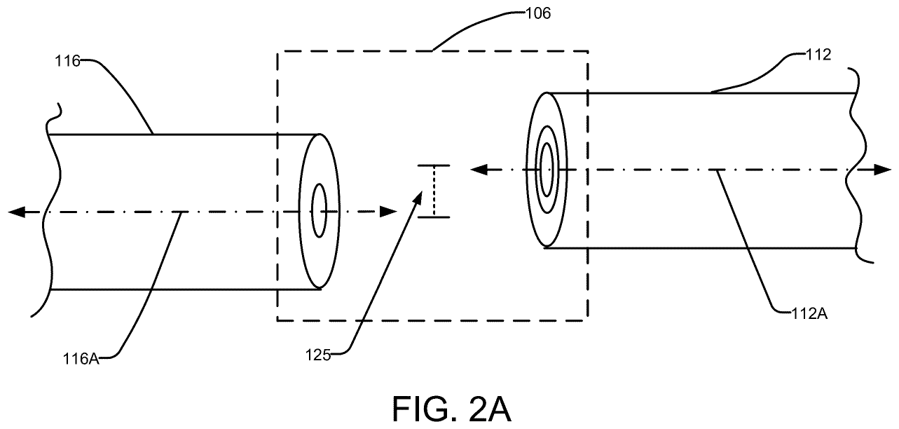

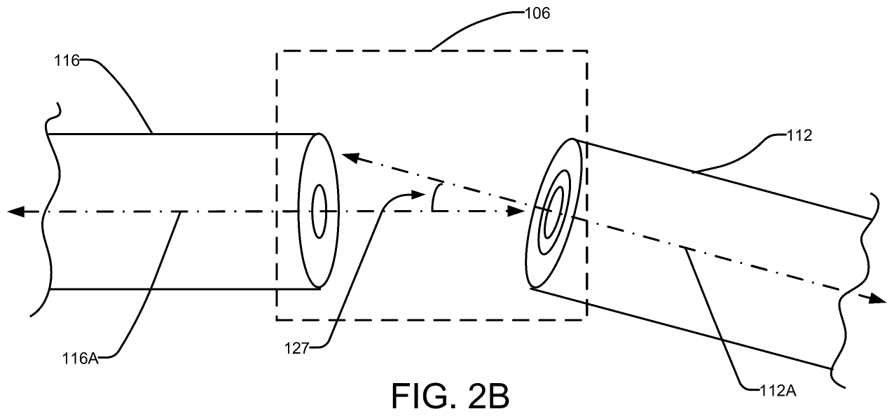

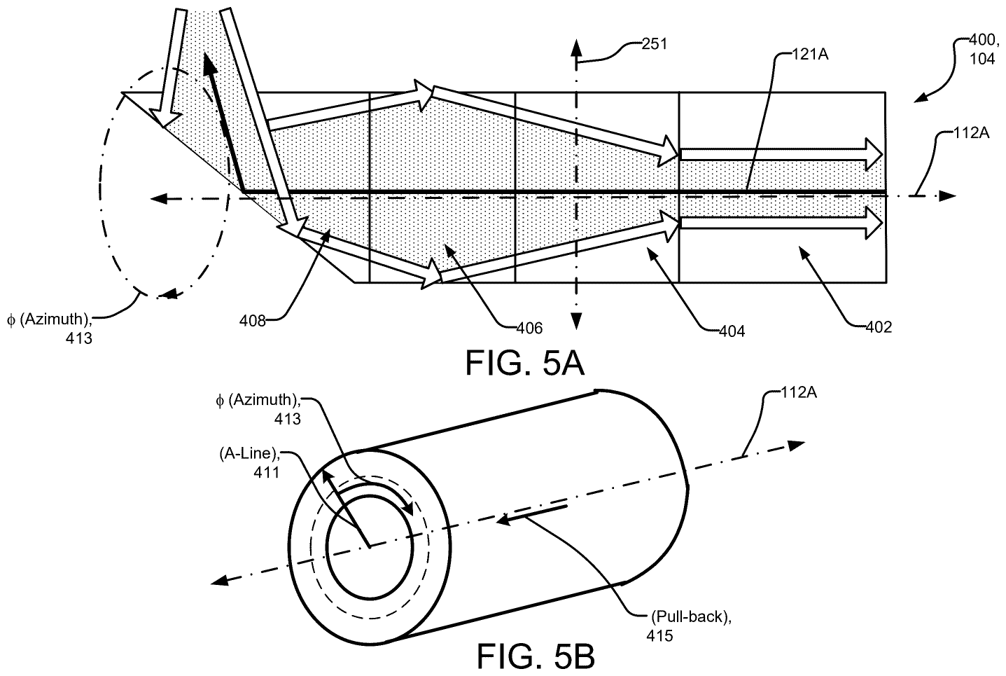

In some embodiments, system 100 comprises a catheter (not expressly shown) in which MCF 112 may be embedded which extends into the body of a patient (sample 111) for in vivo imaging. In some embodiments, optical joint 106 comprises a rotator configured to rotate MCF 112 to scan in azimuthal directions about a pullback axis 415 (described below in relation to FIG. 5B) which may be generally co-linear or parallel with axis 112A of MCF 112 (also described below in relation to FIGS. 2A and 2B). Incident light propagating in MCF 112 and incident light 121A that interacts with sample 111 (and incident light propagating in MCF 112) may comprise a plurality of components; specifically, this incident light may comprise fundamental mode incident light (which may propagate primarily in the core of MCF 112) and higher order mode incident light (which may propagate primarily in the cladding of MCF 112, but which may additionally or alternative comprise core mode(s)). These two components of incident light 121A (and incident light propagating in MCF 112) may arise because of optical joint 106, which may cross-couple some light energy from the fundamental mode of incident light from OCT fiber 116 into higher order mode incident light in MCF 112 (e.g. due to an offset between OCT fiber 116 and MCF 112 as explained in more detail below). This is not necessary, however. There may be a phase delay (positive or negative) between the two components of incident light in MCF 112 due to the different rates of propagation of the two components in MCF 112.

In some embodiments, MCF 112 is capable of receiving high numerical aperture (NA) light—i.e. high NA light may be coupled into MCF 112 for propagation (e.g. multimode propagation) therein. For example, in some embodiments, MCF 112 is capable of receiving light with NA values in the range of up to about 0.2, 0.3 and/or higher. In some embodiments, high NA light received in MCF 112 is propagated in MCF 112 in multiple modes which include a fundamental mode which propagates primarily in a core (not explicitly enumerated) of MCF 112 and one or more higher order mode(s) which propagate primarily in one or more claddings of MCF 112.

Incident light 121A from MCF 112 is directed by sampling optics 104 to impinge onto, and interact with, sample 111. MCF 112 receives reflected/backscattered light 121B from sample 111 via sampling optics 104. This reflected/backscattered light that has interacted with sample 111 may be referred to herein as sampled light. The sampled light 121B received by MCF 112 may also comprise high NA light. The high NA sampled light received by MCF 112 may be propagated back in multiple modes by MCF 112 toward optical joint 106 and OCT system 102. The multiple modes of sampled light propagating in MCF 112 may comprise a fundamental mode (referred to herein as a fundamental MCF mode) and one or more higher order modes (referred to herein a higher order MCF mode(s)). As with the incident light propagating in MCF 112, the higher order MCF mode(s) of sampled light may have a plurality of components which include higher order MCF mode sampled light energy corresponding to fundamental mode incident light that has interacted with sample 111 and higher order MCF mode sampled light energy corresponding to higher order mode incident light that has interacted with sample 111. As is known in the art, MCF 112 has the property that there is relative phase delay between the fundamental MCF mode and each of the higher order MCF mode(s) of sampled light propagating in MCF 112—i.e. sampled light in different modes propagates at different speeds along MCF 112. A total amount of phase delay between MCF modes of sampled light in MCF 112 will also depend on the length of MCF 112 in sample arm 104. It will be appreciated that where the component of higher order MCF mode sampled light energy corresponds to higher order mode incident light, it will have twice the phase delay (positive or negative relative to the fundamental MCF mode sampled light) of the component of higher order MCF mode sampled light energy corresponding to fundamental mode incident light, since the former propagates in higher order modes of MCF 112 in both directions, whereas the latter propagates in the fundamental mode of MCF 112 in the forward direction (toward sample 111) and in the higher order modes of MCF 112 in the reverse direction (away from sample 111).

The high NA sampled light may comprise sampled light backscattered from sample 111 at relatively low angles which propagates primarily in the fundamental MCF mode and primarily in the core of MCF 112 and sampled light backscattered from sampled 111 at relatively large angles which propagates primarily in the higher order MCF mode(s) and primarily in the one or more cladding(s) of MCF 112.

Upon propagating back to optical joint 106 in MCF 112, sampled light propagating in MCF 112 is coupled back into OCT fiber 116 by optical joint 106. Joint 106 is configured to couple at least some of the light energy from both the fundamental MCF mode and the higher order MCF mode(s) into a fundamental mode of OCT fiber 116, where the at least some light energy propagates (back toward interferometric detector 118). The at least some light energy from both the fundamental MCF mode and the higher order MCF mode(s) that is coupled into the fundamental mode of OCT fiber 116 and that propagates back toward interferometric detector 118 may be referred to herein as OCT return light.

The coupling of at least some light energy from the higher order MCF mode(s) into the core of OCT fiber 116 may be referred to herein as cross-coupling. The light cross-coupled from the higher order MCF mode(s) into the core of OCT fiber 116 as OCT return light may comprise some of both components of the higher order MCF mode(s)—i.e. higher order MCF mode sampled light energy corresponding to higher order mode incident light and higher order MCF mode sampled light energy corresponding to fundamental mode incident light. Optical joint 106 may be configured in any suitable manner to cross-couple at least some light energy from higher order MCF mode(s) from MCF 112 into the fundamental mode of OCT fiber 116. In some embodiments, optical joint 106 is provided by spatially and/or angularly offsetting OCT fiber 116 from MCF 112 to enable the cross-coupling.

FIG. 2A is a schematic view of optical joint 106 according to an example embodiment, where OCT fiber 116 is spatially offset from MCF 112. More specifically, in the FIG. 2A embodiment, OCT axis 116A of OCT fiber 116 is spatially offset from MCF axis 112A of MCF 112 by a spatial offset 125. FIG. 2B is a schematic view of optical joint 106 according to another example embodiment, where OCT fiber 116 is angularly offset from MCF 112. More specifically, in the FIG. 2B embodiment, OCT axis 116A of OCT fiber 116 is angularly offset from MCF axis 112A of MCF 112 by an angular offset 127. As discussed above, optical joint 106 may comprise a fiber-optic rotary joint (FORJ). Details of the rotational aspects of optical joint 106 are omitted from FIGS. 2A and 2B.

In some embodiments, OCT fiber 116 is offset by a combination of spatial and angular offset with respect to MCF 112. In some embodiments, other techniques may be used to effect the coupling of at least some of the light energy from the fundamental MCF mode from MCF 112 into the fundamental mode of OCT fiber 116 and cross-coupling at least some light energy from the one or more higher order MCF mode(s) from MCF 112 into the fundamental mode of OCT fiber 116. By way of non-limiting example, such techniques may include: introducing one or more optical elements (e.g. one or more annuli, lenses, mirrors, mediums that cause refraction and/or the like) between MCF 112 and OCT fiber 116 to induce spatial/angular offsets or to otherwise affect the quality or shape of the sampled light emerging from MCF 112 before it enters OCT fiber 116; twisting and/or bending one or both of MCF 112 and OCT fiber 116; applying a mode scrambler to MCF 112 to scramble the higher order MCF mode(s) into the fundamental mode of OCT fiber 116; inducing imperfections in one or both of MCF 112 and OCT fiber 116 (e.g. by pulling, heating, cleaving MCF 112 and/or OCT fiber 116 at an angle, and/or the like); and/or the like.

Returning to FIG. 1, OCT return light (i.e. having at least some light energy coupled from the fundamental MCF mode and the one or more higher order MCF mode(s) in MCF 112 into the fundamental mode of OCT fiber 116 by joint 106) propagates back to OCT system 102, where OCT system 102 is configured to use the OCT return light to generate OCT image data 113. OCT image data 113 may comprise volumetric (e.g. tomographic) data related to sample 111. In some embodiments, the OCT return light is directed to interferometric detector 126 which is configured to generate corresponding OCT image data 113 in accordance with any suitable OCT technique(s) known to those skilled in the art or subsequently developed in the art. In some embodiments, interferometric detector 126 may first generate interferometric data (not expressly shown in FIG. 1) based on the OCT return light and may comprise a suitably configured processor of any suitable type for processing the interferometric data to generate OCT image data 113 according to techniques known to those skilled in the art.

In some embodiments, interferometric detector 126 may generate interferometric data (not expressly shown in FIG. 1) and a controller 120 of imaging system 100 (which may be, in whole or in part, external to OCT system 102 or integrated, in whole or in part, within OCT system 102) may be used to generate OCT image data 113 based on the interferometric data. Controller 120 should be understood to receive OCT image data 113 whether controller 120 is involved in generating OCT image data 113 or receives OCT image data from some separate system (e.g. OCT system 102). In some embodiments, controller 120 comprises one or more stand-along data processors and/or a stand-alone computer. In some embodiments, controller 120 comprises one or more data processors that are integrated into imaging system 100. In general, however, controller 120 may comprise any suitably configured data processor as described elsewhere herein.

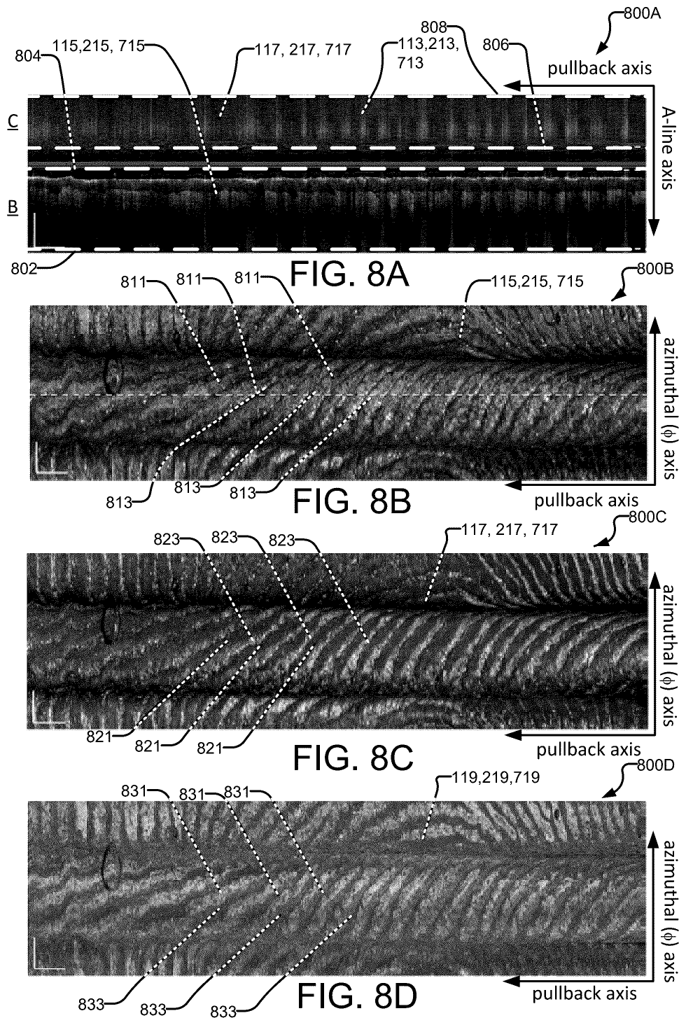

Whether OCT image data 113 is output from interferometric detector 126 and provided to controller 120, or controller 120 is involved in the generation of OCT image data 113, controller 120 receives OCT image data 113. FIG. 8A shows a so-called longitudinal section comprising a pullback direction by A-line slice 800A of exemplary OCT image data 113 corresponding to one azimuthal (ϕ) coordinate (i.e. one azimuthal value (ϕ) about pullback axis 415). Because of the rotation of MCF 112 about its MCF axis 112A (which may be generally coincident or generally parallel with) pullback axis 415, OCT image data 113 comprises a plurality of sections similar to section 800A (each having a different azimuthal coordinate (ϕ)).

It can be observed from the particular example of FIG. 8A, that there are two regions (region B and region C) of bright OCT image data 113 in slice 800A and that these regions are separated from one another in the A-line direction. That is, bright region B generally has higher A-line coordinates than bright region C (In FIG. 8A, the origin of the A-line axis is at the top of the image and the A-line coordinate value increases towards the bottom of the page (as indicated by the arrow of A-line axis) in accordance with customary practice in the OCT field.). Bright regions B and C in OCT image data 113 are created by the different phase delays (introduced in MCF 112) between the fundamental MCF mode light and the higher order MCF mode light. As discussed above, both the fundamental MCF mode and the higher order MCF mode(s) have some light energy coupled into the fundamental mode of OCT fiber 116 as OCT return light. Region B in the FIG. 8A slice 800A of OCT image data 113 may correspond generally to OCT image data 113 associated with the fundamental MCF mode that is coupled into the fundamental mode of OCT fiber 116 and region C in the FIG. 8A slice 800A of OCT image data 113 may correspond to generally to OCT image data 113 associated with the higher order MCF mode(s) that is cross-coupled into the fundamental mode of OCT fiber 116. The difference in the A-line coordinates of regions B and C may be “tuned” by controlling the total phase delay between the fundamental MCF mode light and the higher order MCF mode light in MCF 112 (e.g. by controlling the length and/or other propagation parameter(s) of MCF 112). In some instances, region C may appear to translate with respect to region B when a sample arm position of system 100 is adjusted or if the spatial interferogram is sampled at a different frequency. Nonetheless, region C will generally be present in the reconstructed A-line due to aliasing. While not shown in the exemplary data of FIG. 8A, in circumstances where light cross-coupled from the higher order MCF mode(s) into the core of OCT fiber 116 as OCT return light comprises some of both components of the higher order MCF mode(s)—i.e. higher order MCF mode sampled light energy corresponding to higher order mode incident light and higher order MCF mode sampled light energy corresponding to fundamental mode incident light, the FIG. 8A slice 800A of OCT image data (and similar slices at other azimuthal coordinates (ϕ)) may exhibit a third region of image data in a range of A-line coordinates corresponding to twice the phase delay of region C. This third region of OCT image data (with the largest phase delay—not shown in FIG. 8A) may comprise image data related to the component of higher order MCF mode sampled light energy corresponding to higher order mode incident light, whereas region C shown in FIG. 8A may comprise image data related to the component of higher order MCF mode sampled light energy corresponding to fundamental mode incident light. This OCT image data related to the component of higher order MCF mode sampled light energy corresponding to higher order mode incident light may be referred to herein as additional higher order mode image data 123. Additional higher order mode image data 123 may be extracted from OCT image data 113 in a manner similar to higher order mode image data 117 (e.g. based on A-line coordinate thresholds). Multipath image data 119 may be based on fundamental mode image data 115 and either or both of higher order mode image data 117 and additional higher order mode image data 123.

As explained in more detail below, controller 120 processes OCT image data 113 to: extract fundamental mode image data 115 and higher order mode image data 117 from OCT image data 113; and generate multipath image data 119 based on both fundamental mode image data 115 and higher order mode image data 117.

Extracting fundamental mode image data 115 and higher order mode image data 117 from OCT image data 113 may be based on the A-line coordinates of OCT image data 113. For example, referring to the FIG. 8A pullback by A-line slice 800A of OCT image data 113, fundamental mode image data 115 may correspond to region B and may be extracted by setting lower and upper A-line coordinate thresholds 804, 802—that is fundamental mode image data 115 may correspond to OCT image data 113 between lower A-line coordinate 804 and upper A-line coordinate 802. Similarly, extracting higher order mode image data 117 from OCT image data 113 may be based on lower and upper A-line coordinate thresholds 808, 806. A-line coordinate thresholds 802, 804, 806, 808 are configurable parameters of imaging system 100 and may depend on optical path length (e.g. total phase delay) differences between the fundamental MCF mode light and the higher order MCF mode light introduced by MCF 112.

The inventors have discovered that higher order mode image data 117 may be used in conjunction with fundamental mode image data 115 to generate useful image data referred to herein as multipath image data 119. As explained in more detail below, in some embodiments, generating multipath image data 119 based on both fundamental mode image data 115 and higher order mode image data 117 comprises: generating en face projections corresponding to fundamental mode image data 115 and higher order mode image data 117; and determining multipath image data 119 to be a ratio of the en face fundamental mode image data projection to the en face higher order mode image data projection. This is not necessary. In some embodiments, other techniques based on both fundamental mode image data 115 and higher order mode image data 117 may be used to generate multipath image data 119.

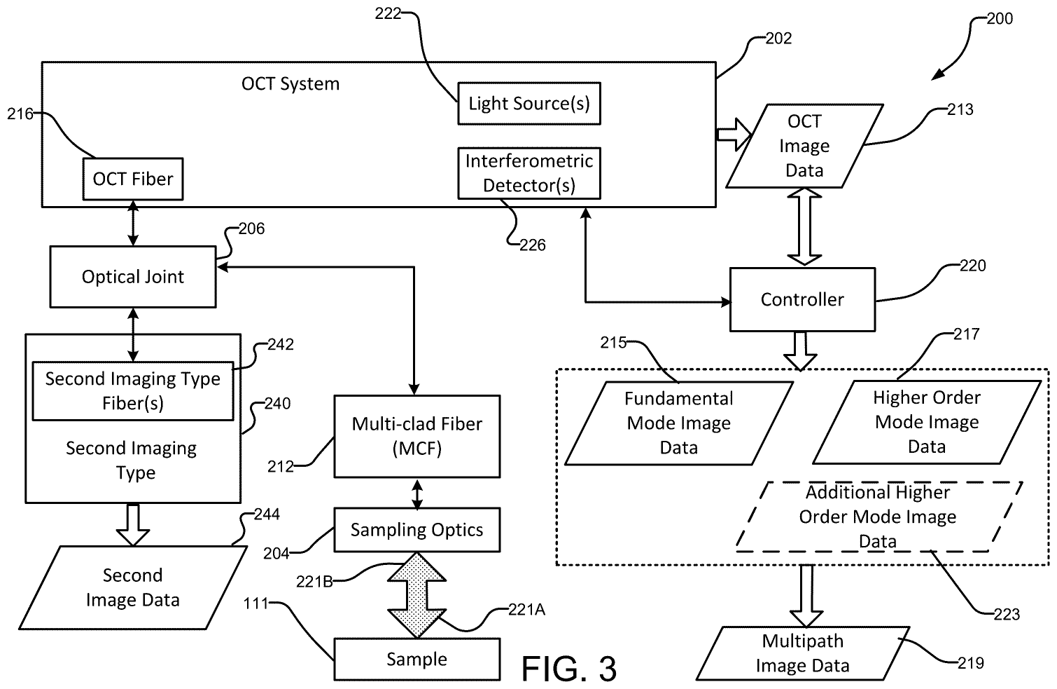

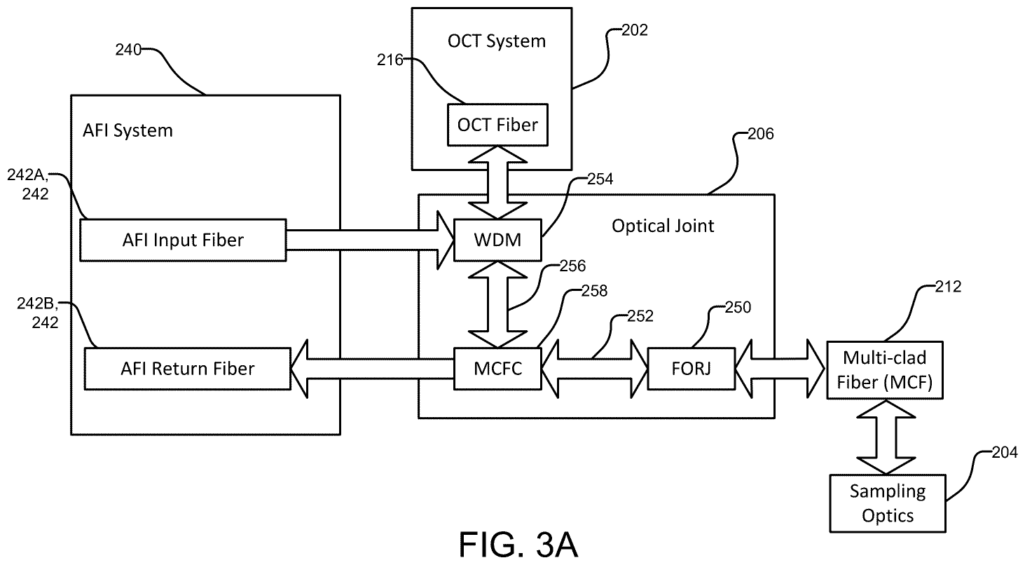

FIG. 3 is a schematic block diagram of a fiber optic imaging system (e.g. an endoscopic system) 200 according to another exemplary embodiment. In many respects, imaging system 200 is similar to imaging system 100 described elsewhere herein and elements and features of imaging system 200 are described using reference numerals that are similar to the reference numerals used in connection with imaging system 100 except that reference numerals of imaging system 200 are preceded by the digit “2” rather than “1” as is the case for features of imaging system 100. For brevity, this description focusses on features of imaging system 200 that are different from those of imaging system 100, it being understood that, unless the context dictates otherwise, features of imaging system 200 are analogous to those of imaging system 100. Imaging system 200 differs from imaging system 100 in that imaging system 200 incorporates two imaging types provided by OCT system 202 and second imaging type 240.

In imaging system 200, OCT imaging system 202 and sampling optics 204 are substantially similar to OCT imaging system 102 and sampling optics 104 of imaging system 100. Imaging system 200 is also similar to imaging system 100 in the manner that: sampled light which has interacted with sample 111 propagates back through MCF 212 (as fundamental MCF mode and one or more higher order MCF mode(s), which may have two components corresponding to higher order mode incident light and corresponding to fundamental mode incident light) and through optical joint 206 which couples at least some of the light energy from both the fundamental MCF mode and the higher order MCF mode(s) into the fundamental mode of OCT fiber 216 (as OCT return light); and OCT return light propagates back to OCT system 202, where interferometric detector 226 generates OCT image data 213 (based on the OCT return light) and controller 220 processes OCT image data 213 to extract fundamental mode image data 215 and higher order mode image data 217 and to generate multipath image data 219 based on both fundamental mode image data 215 and higher order mode image data 217.

Imaging system 200 differs from imaging system 100 in that imaging system 200 comprises a second imaging type provided by second imaging system 240 which: directs second imaging light through second imaging type fiber(s) 242 and optical joint 206, into sampling optics 204 (and MCF 212); receives second imaging type return light via second imaging type fiber(s) 242; and generates second imaging type image data (e.g. second imaging type diagnostic image data) 244 based on the second imaging type return light. More specifically, second imaging system 240 comprises a second light source (not expressly shown in FIG. 3) which directs second incident light through second imaging type fiber(s) 242, whereupon the second incident light is coupled into sampling optics 204 (and MCF 212) by optical joint 206. Light propagates in sampling optics 204 (and MCF 212) in a similar manner to the light of system 100, impinges on (and interacts with) sample 111 and high NA sampled light is coupled back into MCF 212 as fundamental MCF mode and one or more higher MCF modes. At optical joint 206, at least some of the light energy from both the fundamental MCF mode and the higher order MCF mode(s) is coupled into the fundamental mode of OCT fiber 216 (as OCT return light), which is used to generate multipath image data 219 in a manner similar to imaging system 100. However, optical joint 206 of the FIG. 3 system 200 also splits the light from the two imaging types, so that at least some of the light energy from the higher order MCF mode(s) is coupled into second imaging type fiber(s) 242 as second imaging type return light.

Second imaging system 240 receives the second imaging type return light from second imaging type fiber(s) 242 and uses the second imaging type return light to generate second image data 244. In some embodiments, second imaging system 240 may comprise a suitably configured processor of any suitable type for processing the second imaging type return light to generate second image data 244 according to techniques known to those skilled in the art. In some embodiments, controller 220 of imaging system 200 (which may be external to OCT system 202 and second imaging system 240) may be used to generate second image data 244 based on data obtained by second imaging system 240 from the second imaging type return light. In some embodiments, controller 220 comprises a standalone computer. In some embodiments, controller 220 comprises one or more data processors that are integrated into imaging system 200. In general, however, controller 220 may comprise any suitably configured data processor as described elsewhere herein.

In some non-limiting embodiments, second imaging system 240 comprises an autofluorescence imaging (AFI) system 240. In some such embodiments, second imaging type fiber(s) 242 comprise a first (input) fiber 242A which provides second imaging type (AFI) incident light to optical joint 206, MCF 212 and sampling optics 204 and a second (return) fiber 242B which receives second imaging type (AFI) return light from sampling optics 204, MCF 212 and optical joint 206. This is not necessary. In some embodiments, a single second imaging type fiber 242 carries both second imaging type incident light and second imaging type return light.

As alluded to above optical joint 206 of the illustrated FIG. 3 imaging system 200 is similar to optical joint 106 of the FIG. 1 imaging system 100 in that optical joint 206 couples at least some of the light energy from both the fundamental MCF mode and the higher order MCF mode(s) into the fundamental mode of OCT fiber 216 (as OCT return light), which is used to generate multipath image data 219 in a manner similar to imaging system 100. However, optical joint 206 of the FIG. 3 imaging system 200 differs from optical joint 106 of the FIG. 1 imaging system in that optical joint 206 also splits the light from the two imaging types, so that at least some of the light energy from the higher order MCF mode(s) is coupled into second imaging type fiber(s) 242 as second imaging type return light.

FIG. 3A schematically illustrates an optical joint 206 according to a non-limiting example embodiment. In the FIG. 3A embodiment, second imaging system 240 comprises an AFI system 240, where second imaging type fiber(s) 242 comprise a first (input) fiber 242A which provides second imaging type (AFI) incident light to optical joint 206 and a second (return) fiber 242B which receives second imaging type (AFI) return light from optical joint 206. Like optical joint 106 described above, optical joint 206 comprises a FORJ 250 which performs the function of coupling light into and out from MCF 212 and sampling optics 204. FORJ 250 may perform this coupling between MCF 212 and another intermediate MCF 252 which carries both incident AFI and OCT light and AFI return and OCT return light. FORJ 250 may be configured (e.g. to provide spatial and/or angular offset between MCF 212 and intermediate MCF 252 and/or otherwise configured) in a manner similar to that of optical joint 106 described herein to couple at least some light energy from the fundamental MCF mode and the higher order MCF mode(s) from MCF 212 into the fundamental mode of intermediate MCF 252. FORJ 250 may also be configured to couple at least some light energy from the higher order MCF mode(s) from MCF 212 into cladding layers of intermediate MCF 252 which support higher order mode propagation.

Apart from FORJ, optical joint 206 of the FIG. 3A embodiment also comprises a wavelength division multiplexer (WDM) which multiplexes and de-multiplexes light of different wavelengths and a multi-clad fiber coupler (MCFC) which couples light from an input fiber into a multi-clad fiber and back from the multi-clad fiber back into an output fiber. Optical joint 206 functions as follows.

Incident OCT from light source 222 (FIG. 3) of OCT system 202 arrives at WDM 254 on OCT fiber 216. Similarly, incident AFI light from the light source of AFI system 240 arrives at WDM 254 on AFI input fiber 242A. In currently preferred embodiments, OCT fiber 216 comprises a SMF. As discussed above, this is not necessary, and OCT fiber 216 may comprise a SMF, DCF, MCF or FMF. Similarly, AFI input fiber 242A may comprise a SMF, DCF, MCF or FMF. However, the AFI light and OCT light may have different wavelengths so that light from both OCT fiber 216 and AFI input fiber 242A may be multiplexed onto fiber 256 by WDM 254. Fiber 256 may also comprise a SMF, DCF, MCF or FMF. The combined (multiplexed) AFI and OCT incident light propagates on fiber 256 to MCFC 258 which couples the combined incident light onto intermediate MCF 252. MCFC may be configured couple the combined incident light into the core of intermediate MCF 252. The combined incident light then propagates through intermediate MCF 252 to FORJ 250 where it is coupled into MCF 212 and sampling optics 204 as described elsewhere herein.

Light interacts with sample 111 as described elsewhere herein and returns to FORJ 250 as sample light propagating in MCF 212 as fundamental MCF mode and higher order MCF mode(s), which may have two components corresponding to higher order mode incident light and corresponding to fundamental mode incident light. FORJ 250 is configured to couple sample light into both the core and the cladding(s) of MCF 252. FORJ 250 is configured to couple at least some light energy from the fundamental MCF mode and the higher order MCF mode(s) from MCF 212 into the fundamental mode of intermediate MCF 252. FORJ 250 may also be configured to couple at least some light energy from the fundamental MCF mode and the higher order MCF mode(s) from MCF 212 into cladding layers of intermediate MCF 252 which support higher order mode propagation. Return light from intermediate MCF 252 reaches MCFC 258, where the return light is split. MCFC is configured to couple light having wavelengths of both OCT light source 222 and AFI light source from the cladding layer(s) of intermediate MCF 252 primarily into AFI return fiber 242B which may comprise a multi-mode fiber (MMF) or MCF and to couple light from the core of intermediate MCF 252 primarily into the core of fiber 256. AFI return light on AFI return fiber 242B is detected by an AFI detector (cladding light having wavelength of OCT light source 222 is not detected by AFI detector) and used by AFI system to generate AFI image data 244 as described elsewhere herein. Return light propagating on fiber 256 having wavelengths of both OCT light source 222 and AFI light source passes through WDM 254 and is coupled onto OCT fiber 216 as OCT return light, which is in turn used to generate multipath image data 219 as described elsewhere herein. Return light propagating on fiber 256 having wavelength of AFI light source is not detected by the OCT system (i.e. not interfering in a manner to be detectable by the OCT system).

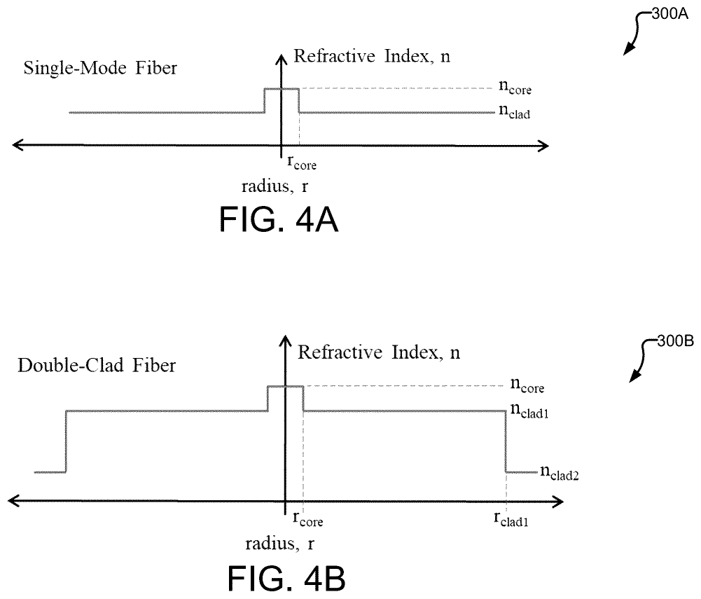

In some embodiments, one or more of the MCFs described herein (e.g. MCF 112, MCF 212, intermediate MCF 252) may comprise double-clad fibers (DCFs). FIGS. 4A and 4B are plots 300A and 300B showing geometric and refractive index properties of SMF and DCF respectively. As shown in FIG. 4A, a SMF has two discrete indices of refraction: a first index of refraction, n_{core}, in its core between the fiber axis and a radial core dimension r_{core}; and a second, lower, index of refraction, n_{clad}, in the radial annular cladding region between the radial core dimension r_{core} and a radius of the protective sheath of the fiber (not shown). In comparison, as shown in FIG. 4B, the DCF of the illustrated embodiment has three indices of refraction: a first index of refraction, n_{core}, in its core between the fiber axis and a radial core dimension r_{core}; a second, lower, index of refraction, n_{clad1}, in an annular cladding region between the radial core dimension r_{core} and a second radial dimension r_{clad1}; and a third, lower still, index of refraction, n_{clad2}, in an annular cladding region between second radial dimension r_{clad1} and a radius of the protective sheath of the fiber (not shown). In some embodiments, a fundamental mode of light energy propagates in the core of a DCF and higher order mode(s) of light energy propagate in the first cladding of the DCF.

In some embodiment, sampling optics (e.g. sampling optics 104 of system 100 and/or sampling optics 204 of system 200) comprises an imaging end 400. Imaging end 400 may comprise any suitable imaging end of any suitable endoscopic imaging system, for example. FIG. 5A shows an enlarged schematic cross-sectional view of an imaging end 400 of sampling optics 104 according to an example embodiment. Sampling optics 204 may comprise a similar imaging end 400. FIG. 5B is a schematic perspective view of a visual representation of the coordinates used to describe OCT image data 113, 213 generated by OCT systems 102, 202 described herein according to an example embodiment.

In the example embodiment shown in FIG. 5A, imaging end 400 comprises a DCF region 402, a multi-mode fiber (MMF) region 404, a gradient index fiber (GRIN) region 406 and an angle-polished no-core fiber (NCF) region 408. However, this is not necessary. Imaging end 400 depicted in the FIG. 5A embodiment represents one particular embodiment for an imaging end. In some embodiments, the imaging systems described herein may use other types of imaging ends which may comprise, by way of non-limiting example, any suitable imaging end of any suitable endoscopic system.

In operation, incident light 121A (illustrated as solid straight arrow in FIG. 5A) is directed onto sample substantially along an A-line axis 411 (shown in FIG. 5B). To perform rotary scanning of a sample 111 (not shown in FIGS. 5A, 5B), sampling optics 104 and MCF 112 including imaging end 400 is rotated by optical joint 106 about MCF axis 112A (pullback axis 415) such that a region of incident light incident on sample 111 continuously traverses to different azimuthal (ϕ) coordinates 413 about MCF axis 112A as MCF 112 is retracted from sample 111 in pullback direction 415 (which may be generally coincident with or generally parallel to MCF axis 112A). The reflected/backscattered (sampled) light (illustrated as white arrows and dotted regions in FIG. 5A) is collected by MCF 112 and propagated through MCF 112 as multiple modes comprising a fundamental MCF mode described herein and one or more higher order MCF modes. Given the retracting rotary scanning, OCT image data 113, 213 may have a helical scan pattern which may be approximated as a three-dimensional cylindrical shape as shown in FIG. 5B.

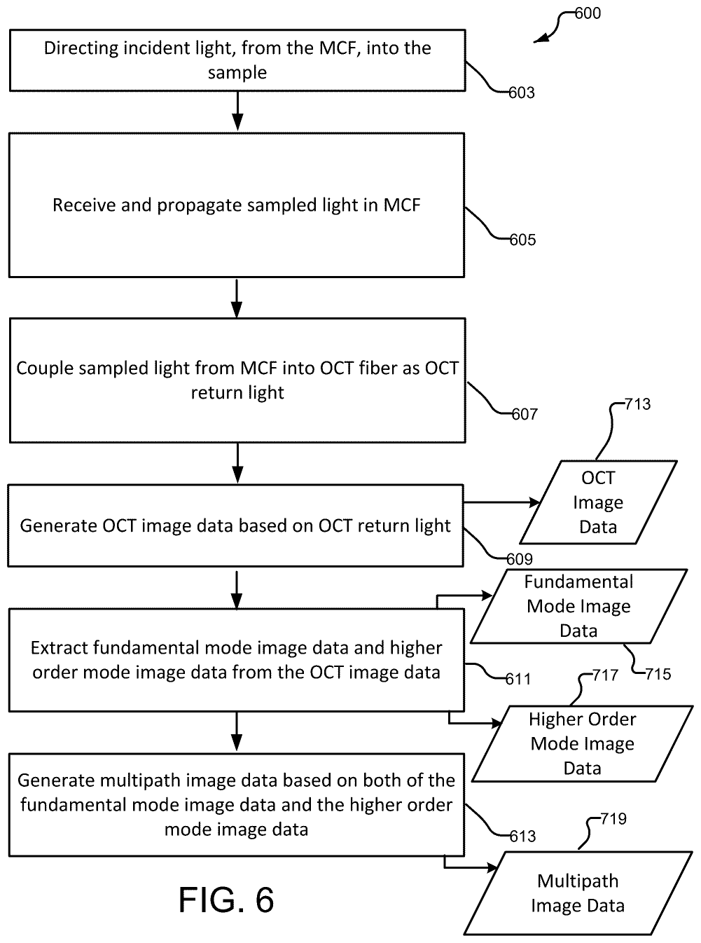

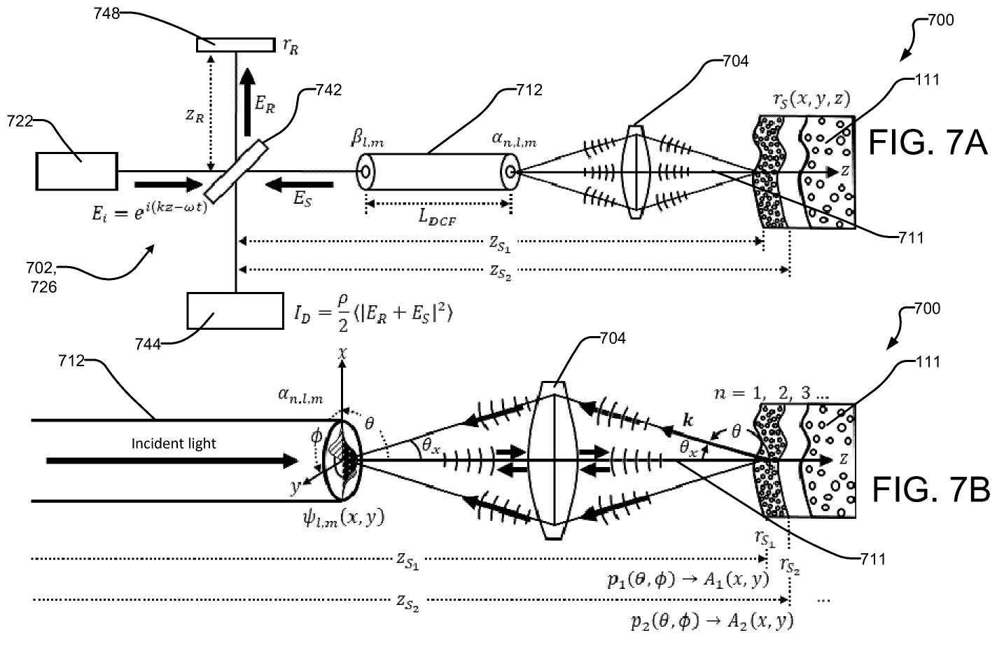

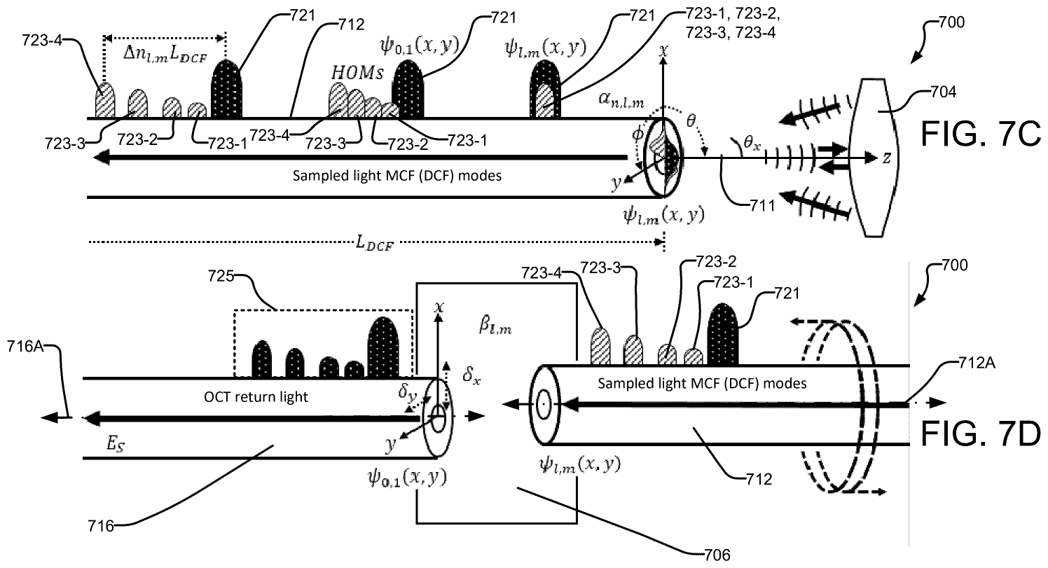

FIG. 6 is a flowchart of a method 600 for generating multipath image data (e.g. multipath image data 119, 219) using an imaging system having a MCF according to an example embodiment. FIG. 6 is described in part in conjunction with FIGS. 7A-7D (collectively, FIG. 7). FIG. 7 includes schematic diagrams of portions of an imaging system 700, illustrating the propagation of light through imaging system 700 according to an example embodiment. Imaging system 700 may be similar to imaging systems 100, 200 described above and reference numerals for features of imaging system 700 that are analogous to features of imaging systems 100, 200 are similar to the reference numerals of imaging systems 100, 200 except that reference numerals of imaging system 700 are preceded by the digit “7” rather than “1” (in the case of imaging system 100) or “2” (in the case of the imaging system 200). Features of imaging system 700 (e.g. particular fibers and/or other optical components) may be used in either of imaging systems 100, 200. For brevity, this description focusses on features of imaging system 700 that are different from those of imaging systems 100, 200 it being understood that, unless the context dictates otherwise, features of imaging system 700 are analogous to those of imaging systems 100, 200. While the FIG. 6 method 600 is described in part in conjunction with imaging system 700, it will be appreciated that method 600 could also be used with the other imaging systems (e.g. imaging systems 100, 200) described herein. Further, the explanation of the FIG. 6 method 600 in conjunction with the FIG. 7 optical system 700 I described with theoretical models of various features of optical system 700. Neither method 600 nor optical system 700 are bound by any of these models.

Method 600 involves collecting image data from a sample using a MCF. The sample may be, but is not limited to, the body of a human or animal patient. The MCF may be any suitable MCF described herein (e.g. MCF 12, 212, 712). In block 603, incident light is directed, from the MCF, into the sample, where it interacts with the sample. The light propagation in block 603 is illustrated in MCF 712 shown in FIG. 7A. In some embodiments, MCF 712 comprises a DCF 712, although this is not necessary and any suitable fiber supporting multimode propagation may be used. FIG. 7A also shows imaging system 700 incorporating an OCT system 702 comprising a Michelson interferometer 726, although this also is not necessary and any suitable interferometry system may be used.

As shown in FIG. 7A, light source 722 emits incident light towards sample 111 along an optical axis 711. In accordance with convention in the OCT field, optical axis 711 may refer to the direction that light is travelling at any point in system 700 and is often assigned the coordinate (z). It will be appreciated that optical axis 711 and/or its corresponding z coordinates may change orientations when the direction that light is travelling in system 700 changes direction. In some embodiments, the incident light may be modelled as a polychromatic plane wave with electric field defined as:

$${E}_{i} = s ( k , ω ) {e}^{i ( kz - ω t )}$$

where s(k, ω) is the electric field amplitude as function of wavenumber k=2π/λ and angular frequency ω=2πv, λ is wavelength, v is frequency and i=\sqrt{−1}.

Incident light from light source 722 impinges on beam splitter 742, a reference reflector 748 and a detector 744. Reference reflector 748 may be modelled as having an electric field reflectivity r_{R}, and power reflectivity R_{R}=|r_{R}|². The reference path of interferometer 726 may be air and the distance from beam splitter 742 to reference reflector 748 may be referred to as z_{R}.

Returning to FIG. 6, in block 603, the incident light is directed by sampling optics 704 from MCF 712 into sample 111, where the incident light interacts with sample 111. In block 605, MCF 712 receives sampled light that has interacted with sample 111 and the sampled light propagates in multiple modes within MCF 712 (e.g. in a fundamental MCF mode and one or more higher order MCF modes). There is a relative phase delay between at least two of the multiple MCF modes propagating in MCF 712. In some embodiments, the multiple modes of the reflected light correspond to the angles of reflection and/or backscattering of light that has interacted with sample 111. For example, higher order MCF mode(s) may correspond primarily to higher angular backscattering and the fundamental MCF mode may correspond primarily to low angle backscattering.

The process of block 605 is shown schematically in exemplary imaging system 700 of FIG. 7B. Sample 111 under interrogation in FIG. 7 may be characterized by a depth- and angular-dependent reflectivity, r_{S}(x, y, z). As illustrated in FIG. 7B, sample 111 may be modelled as having a series of N discrete angle-dependent reflections given by:

$${r}_{S} ( x , y , z ) = {\sum }_{n = 1}^{N} {r}_{{S}_{n}} {A}_{n} ( x , y ) δ ( z - {z}_{{S}_{n}} )$$

with each reflection characterized by its electric field reflectivity [r_{S}_{₁}, r_{S}_{₂}, . . . , r_{S}_{ₙ}] or power reflectivity R_{S}_{ₙ}=|r_{S}_{ₙ}|², path length from beam splitter 742 [z_{S}_{₁}, z_{S}_{₂}, . . . , z_{S}_{ₙ}], and a complex envelope [A₁ (x, y), A₂ (x, y), . . . , A_{N} (x, y)] that encodes the angular distribution of the backscattered light. The Fourier transform of the complex envelope Ã_{N}(kₓ, k_{y}) describes the angular distribution of the backscattered light, where kₓ=kθₓ and k_{y}=kθ_{y} are angular spatial frequencies along the x- and y-axes under the paraxial approximation, θₓ is the angle between the wavevector and the yz-plane, and θ_{y} (not shown in FIG. 7B) is the angle between wavevector and the xz-plane.

When A_{N} (x, y)=1, then Ã_{N} (kₓ, k_{y})=δ(kₓ, k_{y}), and the reflected light consists of backscattered light with scattering angle θ=±180°. Similarly, a reflector with isotropic scattering has A_{N}(x, y)=δ(x, y) and Ã_{N}(kₓ, k_{y})=1. A reflector that backscatters at an angle θₓ<180° to an optical axis 711 will have A_{N}(x, y)=e^{ikθ}^{ₓ}^{x} and Ã_{N}(kₓ, k_{y})=δ(kₓ−kθₓ, k_{y}). The complex envelope A_{N}(x, y) corresponding to an arbitrary scattering phase function pₙ(θ) can be determined by first translating the coordinate axis by 180° to bring backscattered light to the origin then taking the Fourier transformation of the resulting phase function.

In the FIG. 7 embodiment, incident light is propagated in the fundamental mode of MCF 712 and projected into sample 111 by sampling optics 704 (shown in FIGS. 7A-C). Azimuthally symmetric (the azimuthal coordinate denoted as ø in FIG. 7B) sampled light backscattered along optical axis 711 is coupled into the fundamental mode of MCF 712 (fundamental MCF mode or, in the case of DCF 712, fundamental DCF mode 712). Sampled light backscattered at a non-zero angle θ to optical axis 711 may only be partially coupled into the fundamental mode, with some fraction of the optical power coupled into one or more higher order modes of MCF 712 (higher order MCF mode(s) or, in the case of DCF 712, higher order DCF mode(s)).

The power fraction coupled into a particular mode depends on its spatial overlap with the incident electric field A_{N}(x, y). Using an overlap integral, the power coupled from discrete reflection n into linearly polarized mode LPₗₘ of MCF 712 may be modelled as:

$${α}_{n , l , m} = \frac{{\vert \int \int {A}_{N} ( x , y ) {ψ}_{l , m}^{*} ( x , y ) dxdy \vert}^{2}}{\int \int {\vert {\tilde{A}}_{N} ( x , y ) \vert}^{2} dxdy \int \int {\vert {ψ}_{l , m}^{*} ( x , y ) \vert}^{2} dxdy} \qquad ( 1 )$$

where ψ_{l,m} is the transverse mode profile of LPₗₘ, l and m are the azimuthal and radial mode orders, and the integration is over the end face of MCF 712.

Equivalently, the coupling coefficient can also be calculated in the spatial frequency domain:

$${α}_{n , l , m} = \frac{{\vert \int \int {\tilde{A}}_{N} ( {k}_{x} , {k}_{y} ) {\tilde{ψ}}_{l , m}^{*} ( {k}_{x} , {k}_{y} ) {dk}_{x} {dk}_{y} \vert}^{2}}{\int \int {\vert {\tilde{A}}_{N} ( {k}_{x} , {k}_{y} ) \vert}^{2} {dk}_{x} {dk}_{y} \int \int {\vert {\tilde{ψ}}_{l , m}^{*} ( {k}_{x} , {k}_{y} ) \vert}^{2} {dk}_{x} {dk}_{y}} \qquad ( 2 )$$

where \tilde{ψ}_{l,m}(kₓ, k_{y}) is the Fourier transform of the transverse mode profile and the integration is over the angles supported by the aperture of sampling optics 704.

Ideal backscatter (θ=±180°, θₓ=θ_{y}=0) is coupled almost entirely into the fundamental mode and thus the coupling coefficient as defined above is close to one. Sampled light that is not as directly backscattered may partially couple into the fundamental mode and one or more higher order modes as characterized by α_{n,l,m}. This angle-dependent mode coupling provides a means to differentiate scatterers via scattering angle distribution and may allow elucidation of the scattering angle distribution of the sampled light backscattered from a reflector.

Higher order modes with a non-zero azimuthal mode order (e.g. LP₁₁ in a step-index fiber) are sensitive to non-orthogonal incident light (θₓ, θ_{y}>0) only if the reflector is displaced some small distance (δₓ, δ_{y}) from the optical axis 711. Consequently, if such a reflector is on axis (δₓ=δ_{y}=0), the overlap integral given by equations. (1) and (2) is zero. In a scanning system, this means maximum sensitivity to a reflector with angle-dependent backscatter will occur when the reflector is slightly offset from optical axis 711. To accommodate for this offset required for sensitivity, the mean coupling coefficient may be used:

$${\tilde{α}}_{n , l , m} = \frac{{\vert \int \int {\tilde{A}}_{N} ( {k}_{x} , {k}_{y} ) {\tilde{ψ}}_{l , m}^{*} ( {k}_{x} , {k}_{y} ) \vert}^{2} {dk}_{x} {dk}_{y}}{\int \int {\vert {\tilde{A}}_{N} ( {k}_{x} , {k}_{y} ) \vert}^{2} {dk}_{x} {dk}_{y} \int \int {\vert {\tilde{ψ}}_{l , m}^{*} ( {k}_{x} , {k}_{y} ) \vert}^{2} {dk}_{x} {dk}_{y}} \qquad ( 3 )$$

as a surrogate for the ideal coupling coefficient defined above. The absolute square now operates directly on the Fourier transform of the complex envelope and transverse profile rather than the integral. This is equivalent to integrating over the offset-dependent coupling coefficient ã_{n,l,m}(δₓ, δ_{y}) and effectively removes the offset dependency from the coupling coefficients.

Referring to FIG. 7C, an angle-dependent reflection from each discrete reflector is decomposed by MCF 712 into a set of LPₗₘ MCF modes 721 and 723-1 to 723-4 (or in the case of DCF 712, DCF modes) which propagate down MCF 712. Each MCF mode (e.g. fundamental MCF mode 721 and higher order MCF modes 723-1 to 723-4 shown in FIG. 7C) propagates with a unique effective refractive index and phase velocity resulting in relative phase delay between the different MCF modes. Higher order MCF modes 723-1 to 723-4 with effective refractive indices less than the fundamental MCF mode 721 will propagate faster and appear closer to the zero-reference delay in a processed OCT image, permitting modes which correspond to higher-angle backscatter to be identified given sufficient phase delay.

Returning to FIG. 6, in block 607, the sampled light travelling in the MCF (as fundamental MCF mode (e.g. mode 721) and one or more higher order MCF modes (e.g. modes 723-1 to 723-4)) is directed to an optical joint 706, which is configured to couple at least some light energy from both the fundamental MCF mode 721 and the one or more higher order MCF modes 723-1 to 723-4 from MCF 712 into a fundamental mode 725 of a fiber 716, where the at least some light energy propagates as OCT return light. In some embodiments, fiber 716 is similar to OCT fiber 116 in that it is coupled directly to the OCT system. In some embodiments, fiber 716 is similar to intermediate fiber 252 that is coupled to a fiber coupler in the optical joint and indirectly coupled to the OCT system. Optical joint 706 is configured in any suitable manner to cross-couple at least some light energy from higher order MCF mode(s) 723-1 to 723-4 from MCF 712 into the fundamental mode 725 of fiber 716. This block 607 process is shown schematically in FIG. 7D, where optical joint 706 comprises a FORJ.

Referring to FIG. 7D, optical joint 706 is configured to couple at least some light energy from the now-dispersed (because of phase delay) higher order MCF mode(s) 723-1 to 723-4 back into the fundamental mode 725 of fiber 716 (as OCT return light) which is directed to interferometer 726 to interfere with light from reference reflector 748. Optical joint 706 may be configured to provide a slight offset (δₓ, δ_{y}) between the axes 712A, 716A of MCF 712 and fiber 716. The overlap integral:

$${β}_{l , m} = \frac{{\vert \int \int {ψ}_{0 , 1} ( x , y ) {ψ}_{l , m}^{*} ( x \pm {δ}_{x} , y \pm {δ}_{y} ) dxdy \vert}^{2}}{\int \int {\vert {ψ}_{0 , 1} ( x , y ) \vert}^{2} dxdy \int \int {\vert {ψ}_{l , m}^{*} ( \pm {δ}_{x} , y \pm {δ}_{y} ) \vert}^{2} dxdy} \qquad ( 4 )$$

may be used to characterize the power coupling from LPₗₘ to LP₀₁, where the spatial-domain integration is over the end face of fiber 716.

In block 609, the OCT return light propagating in fiber 716 is directed to interferometer 726 to thereby generate OCT image data 713.

Referring to FIG. 7, the electric field incident on beam splitter 742 and returning from reference reflector 748 is

$${E}_{R} = \frac{{E}_{i}}{\sqrt{2}} {r}_{R} {e}^{i 2 {kz}_{R}} ,$$

while that the electric field returning from fiber 716 is:

$${E}_{S} = \frac{{E}_{i}}{\sqrt{2}} {\sum }_{n = 1}^{N} {\sum }_{l = 0}^{L} {\sum }_{m = 1}^{M} \sqrt{{R}_{{S}_{n}}} \sqrt{{α}_{n , l , m} {β}_{l , m}} {e}^{ik ( 2 {z}_{{S}_{n}} + Δ {n}_{l , m} {L}_{DCF} )} \qquad ( 5 )$$

The electric field at beam splitter 742 is the sum of all modes and all discrete reflectors. The factor of 2 in the first term of the exponential accounts for the roundtrip path length to each reflection. The second term, Δn_{l,m}L_{DCF}, is non-zero for higher-order modes, reducing the effective path length of the reflection and translating it in the negative z direction. The effective refractive index difference, Δn_{l,m}=n_{l,m}−n_{0,1}, is definitionally negative for higher order modes, where n_{l,m} is the effective refractive index of mode (l, m). The physical length of MCF 712 L_{DCF} is multiplied by the mode-dependent refractive index difference to calculate the phase offset resulting in a translation of the higher order modes reflection in the negative z direction.

Detector 744 generates a photocurrent

$${I}_{D} = \frac{ρ}{2} 〈 {\vert {E}_{R} + {E}_{S} \vert}^{2} 〉$$

proportional to the square of the sums of the reference and sample electric fields, where ρ is the responsivity of detector 744, and the angled brackets represent time averaging. The spectral interferogram contains DC, cross-correlation, and auto-correlation terms. The cross-correlation terms,

$${I}_{OCT} ( k ) = \qquad ( 6 )$$

$$\frac{ρ}{4} S ( k ) \{ \begin{matrix}{\sum }_{n = 1}^{N} \sqrt{{R}_{R} {R}_{{S}_{n}}} \sqrt{{α}_{n , 0 , 1} {β}_{0 , 1}} cos [ 2 k ( {z}_{R} - {z}_{{S}_{n}} ) ] + \\ {\sum }_{n = 1}^{N} {\sum }_{l = 1}^{L} {\sum }_{m = 1}^{M} \sqrt{{R}_{R} {R}_{{S}_{n}}} \sqrt{{α}_{n , l , m} {β}_{l , m}} cos [ k ( 2 ( {z}_{R} - {z}_{{S}_{n}} ) + Δ {n}_{l , m} {L}_{DCF} ) ]\end{matrix} \}$$

encode the position, reflectivity, and angular distribution of the reflectors, where S(k)= [symbol] |s(k, ω)|² [symbol] is the power spectrum of the source. Note that in equation (6), the term owing to the fundamental mode (l=0, m=1) has been written explicitly to distinguish it from the terms responsible for the higher order modes. An inverse Fourier transform can then be applied to recover the A-line profile:

$${i}_{OCT} ( z ) = \frac{ρ}{4} γ ( z ) ⊗ \{ \begin{matrix}{\sum }_{n = 1}^{N} \sqrt{{R}_{R} {R}_{{S}_{n}}} \sqrt{{α}_{n , 0 , 1} {β}_{0 , 1}} δ [ z \pm 2 ( {z}_{R} - {z}_{{S}_{n}} ) ] + \\ {\sum }_{n = 1}^{N} {\sum }_{l = 1}^{L} {\sum }_{m = 1}^{M} \sqrt{{R}_{R} {R}_{{S}_{n}}} \sqrt{{α}_{n , l , m} {β}_{l , m}} δ [ z \pm 2 ( {z}_{R} - {z}_{{S}_{n}} ) + Δ {n}_{l , m} {L}_{DCF} ]\end{matrix} \} \qquad ( 7 )$$

where γ(z) is the coherence (or point spread) function, ⊗ is the convolution operator, and δ is the Dirac delta function.

Cross-coupling by optical joint 706 of at least some light energy from one or more higher order MCF modes 723-1 to 723-4 in MCF 712 into the fundamental mode 725 of fiber 716 as OCT return light is highest when there is a large spatial overlap in the geometric area of the core of the respective fibers 712 and 716. In some embodiments, azimuthal modes of order l=1 are cross-coupled sufficiently (i.e. high β_{l,m}) into fundamental mode 725 of fiber 716 for detection through interference with the reference. As described elsewhere herein, this results in higher order mode image data 717 which is consistently composed of the same higher order modes, and therefore appears in the same location relative to the fundamental mode image data 715 within a given A-line, which permits the extraction of the fundamental mode image data 715 and higher order mode image data 717 from the OCT image data 713 based on A-line coordinates.

Returning to FIG. 6, in block 611, fundamental mode image data 715 and higher order mode image data 717 are extracted from OCT image data 713. The extraction of fundamental mode image data and higher order mode image data is based on A-line coordinates of OCT image data 713 as described elsewhere herein. The block 611 extraction may be performed by a controller (see controllers 120, 220 in FIGS. 1 and 2).

In block 613, the controller generates multipath image data 719 based on both of fundamental mode image data 715 and higher order mode image data 717. In some embodiments, generating multipath image data 719 based on both of fundamental mode image data 715 and higher order mode image data 717 comprises obtaining a ratio between fundamental mode image data 715 and higher order mode image data 717 (i.e. contrast between fundamental mode image data 715 and higher order mode image data 717). In some embodiments, fundamental mode image data 715 comprises a fundamental mode image (FI) and higher order mode image data 717 comprises a higher order mode image (HOI).

In a non-limiting example embodiment, multipath contrast (MC) is obtained to leverage the differential coupling of sampled light into the FI and the HOI. MC is defined as the ratio between the optical powers coupled into the FI versus the HOI, which can be formulated from equation (7) as follows:

$$MC = \frac{{\sum }_{n = 1}^{N} \sqrt{{R}_{{S}_{n}}} \sqrt{{α}_{n , 0 , 1} {β}_{0 , 1}}}{{\sum }_{n = 1}^{N} {\sum }_{l = 1}^{L} {\sum }_{m = 1}^{M} \sqrt{{R}_{{S}_{n}}} \sqrt{{α}_{n , l , m} {β}_{l , m}}} \qquad ( 8 )$$

The MC is a function of the cross-coupling efficiencies β_{l,m} through a discontinuity (optical joint 106, 206, 706) and the FI and HOI coupling efficiencies α_{n,l,m}. Notably, the ratio-metric nature of equation (8) removes any dependency on the reflectivity of the reference arm, thus limiting the variance in MC response from changes in the reference arm position or focus. Assuming the modal cross-coupling efficiencies of optical joint 106, 206, 706 remain constant between images, MC is proportional to the ratio of a backscattered electric field sampled at low angles (by the LP₀₁ mode) over the field sampled at higher angles (HOMs of the form LP₁₁-like).

FIG. 8A is a plan view of a so-called longitudinal section (i.e. a pullback by A-line section) 800A of example OCT image data 113, 213, 713 corresponding to one azimuthal (ϕ) coordinate (i.e. one azimuthal value (ϕ) about pullback axis 415). It will be appreciated that OCT image data 113, 213, 713 comprises a plurality of sections similar to section 800A (each having a different azimuthal coordinate (ϕ)). FIG. 8B is a plan view of an “en face” projection 800B of fundamental mode image data 115, 215, 715 of the exemplary FIG. 8A OCT image data 113, 213, 713 according to an example embodiment. FIG. 8C is a plan view of an en face projection 800C of higher order mode image data 117, 217, 717 of the exemplary FIG. 8A OCT image data 113, 213, 713 according to an example embodiment. FIG. 8D is a plan view of an en face projection 800D of a multipath contrast image based on the FIG. 8B fundamental mode image data 115, 215, 715 and the FIG. 8C higher order mode image data 117, 217, 717 according to an example embodiment.

FIGS. 8A-D (collectively, FIG. 8) are depictions of exemplary data obtained from imaging a human fingertip. Pullback by A-line longitudinal section 800A (FIG. 8A) is shown in a plane defined by an A-line axis (e.g. A-line 411 shown in FIG. 5B) corresponding to one azimuthal (ϕ) coordinate (i.e. one azimuthal value (ϕ) about pullback axis 415) and a pullback axis 415 (see FIG. 5B), which is the direction in which the MCF 112, 212, 712 is pulled back from sample 111. Because of the rotation of MCF 112, 212, 712 about its own axis (which may be coincident or parallel with pullback axis 415), OCT image data 113 comprises a plurality of sections similar to section 800A (each having a different azimuthal coordinate (ϕ)). Therefore, pullback by A-line section 800A may be thought of as one pullback by radial (or pullback by A-line) slice (corresponding to one azimuthal value (ϕ)) of example OCT image data 113, 213, 713, it being understood that there are a plurality of similar pullback by A-line slices (one for each azimuthal value (ϕ)) in OCT image data 113, 213, 713.

It can be observed from FIG. 8A, that there are two regions (region B and region C) of bright OCT image data 113, 213, 713 in slice 800A and that these regions are separated from one another in the A-line direction. That is, bright region B generally has higher A-line coordinates than bright region C. The spatial offset between bright regions B and C in OCT image data 113, 213, 713 is created by the different phase delays (introduced in MCF 112, 212, 712) between the fundamental MCF mode light and the higher order MCF mode light. The unique transverse profile of each mode provides angular sensitivity (i.e. the modes are sensitive to slightly different incident EM fields backscattered from the sample and therefore captures scattering differently). As discussed above, both the fundamental MCF mode and the higher order MCF mode(s) have some light energy coupled into the fundamental mode of OCT fiber 116, 216, 716 as OCT return light. Region B in the FIG. 8A slice 800A of OCT image data 113, 213, 713 may correspond generally to OCT image data 113, 213, 713 associated with the fundamental MCF mode that is coupled into the fundamental mode of OCT fiber 116, 216, 716 and region C in the FIG. 8A slice 800A of OCT image data 113, 213, 713 may correspond to generally to OCT image data 113, 213, 713 associated with the higher order MCF mode(s) that is cross-coupled into the fundamental mode of OCT fiber 116, 216, 716. The difference in the A-line coordinates of regions B and C may be “tuned” by controlling the total phase delay between the fundamental MCF mode light and the higher order MCF mode light in MCF 112, 212, 712 (e.g. by controlling the length and/or other propagation parameter(s) of MCF 112, 212, 712). In some instances, region C may appear to translate with respect to Region B when a sample arm position of system 100 is adjusted or if the spatial interferogram is sampled at a different frequency. Nonetheless, region C will generally be present in the reconstructed A-line due to aliasing. While not shown in the exemplary data of FIG. 8A, in circumstances where light cross-coupled from the higher order MCF mode(s) into the core of OCT fiber 116, 216, 716 as OCT return light comprises some of both components of the higher order MCF mode(s)—i.e. higher order MCF mode sampled light energy corresponding to higher order mode incident light and higher order MCF mode sampled light energy corresponding to fundamental mode incident light, the FIG. 8A slice 800A of OCT image data (and similar slices at other azimuthal coordinates (¢)) may exhibit a third region of image data in a range of A-line coordinates corresponding to twice the phase delay of region C. This third region of OCT image data (with the largest phase delay—not shown in FIG. 8A) may comprise image data related to the component of higher order MCF mode sampled light energy corresponding to higher order mode incident light, whereas region C shown in FIG. 8A may comprise image data related to the component of higher order MCF mode sampled light energy corresponding to fundamental mode incident light. This OCT image data related to the component of higher order MCF mode sampled light energy corresponding to higher order mode incident light may be referred to herein as additional higher order mode image data 123, 223, 723. Additional higher order mode image data 123, 223, 723 may be extracted from OCT image data 113, 213, 713 in a manner similar to higher order mode image data 117, 217, 717 (e.g. based on A-line coordinate thresholds). Multipath image data 119, 219, 719 may be based on fundamental mode image data 115, 215, 715 and either or both of higher order mode image data 117, 217, 717 and additional higher order mode image data 123, 223, 723.

As explained above, OCT image data 113, 213, 713 is processed to: extract fundamental mode image data 115, 215, 715 and higher order mode image data 117, 217, 717 from OCT image data 113, 213, 713 (see block 611 of method 600 in FIG. 6); and generate multipath image data 119, 219, 719 based on both fundamental mode image data 115, 215, 715 and higher order mode image data 117, 217, 717 (see block 613 of method 600 in FIG. 6).

Extracting fundamental mode image data 115, 215, 715 and higher order mode image data 117, 217, 717 from OCT image data 113, 213, 713 may be based on the A-line coordinates of OCT image data 113, 213, 713. For example, referring to the FIG. 8A pullback by A-line slice 800A of OCT image data 113, 213, 713, fundamental mode image data 115, 215, 715 may correspond to region B and may be extracted by setting lower and upper A-line coordinate thresholds 804, 802—that is fundamental mode image data 115, 215, 715 may correspond to OCT image data 113, 213, 713 between lower A-line coordinate 804 and upper A-line coordinate 802. Similarly, extracting higher order mode image data 117, 217, 717 from OCT image data 113, 213, 713 may be based on lower and upper A-line coordinate thresholds 808, 806. A-line coordinate thresholds 802, 804, 806, 808 are configurable parameters of imaging system 100 and may depend on optical path length (e.g. total phase delay) differences between the fundamental MCF mode light and the higher order MCF mode light introduced by MCF 112, 212, 712.

The inventors have discovered that higher order mode image data 117, 217, 717 may be used in conjunction with fundamental mode image data 115, 215, 715 to generate useful image data referred to herein as multipath image data 119, 219, 719. In some embodiments, generating multipath image data 119, 219, 719 based on both based on both fundamental mode image data 115, 215, 715 and higher order mode image data 117, 217, 717 comprises: generating en face projections corresponding to fundamental mode image data 115, 215, 715 and higher order mode image data 117, 217, 717; and determining multipath image data 119, 219, 719 to be a ratio of the en face fundamental mode image data projection to the en face higher order mode image data projection. This is not necessary. In some embodiments, other techniques based on both fundamental mode image data 115, 215, 715 and higher order mode image data 117, 217, 717 may be used to generate multipath image data 119, 219, 719.

FIG. 8B shows an exemplary en face projection 800B of fundamental mode image data 115, 215, 715 extracted from the exemplary OCT image data 113, 213, 713 shown in FIG. 8A (and the additional OCT image data 113, 213, 713 corresponding to other azimuthal (ϕ) coordinates) on the basis of A-line coordinates. FIG. 8A shows an exemplary region B of longitudinal section 800A from which a portion of en face projection 800B can be extracted. Region B of A-line by pullback longitudinal section 800A can be seen to be a region of relatively higher A-line coordinates (when compared to region C of A-line by pullback longitudinal section 800A) between a lower A-line coordinate threshold 804 and an upper A-line coordinate threshold 802. As discussed above, the A-line by pullback longitudinal section 800A of FIG. 8A represents a single azimuthal value (ϕ) in OCT image data 113, 213, 713. The en face projection 800B of FIG. 8B may be obtained by: for each azimuthal coordinate (ϕ) and for each pullback axis coordinate in OCT image data 113, 213, 713 (i.e. for each azimuthal, pullback coordinate pair in OCT image data 113, 213, 713), summing or averaging or otherwise aggregating or processing values along the A-line direction between lower A-line coordinate threshold 804 and upper A-line coordinate threshold 802 (e.g. in region B shown in FIG. 8A) to obtain a value for an en face pixel corresponding to the current azimuthal, pullback coordinate pair. Since the A-line by pullback longitudinal section 800A shown in FIG. 8A represents only one azimuthal value (ϕ) of OCT image data 113, 213, 713, the data of FIG. 8A only creates one horizontal line of pixels in the FIG. 8B en face image 800B. En face image 800B is a projection in the pullback and azimuthal coordinate (ϕ) axes.

Exemplary en face projection 800C of higher order mode image data 117, 217, 717 is extracted from the exemplary OCT image data 113, 213, 713 on the basis of A-line coordinates in a manner similar to en face projection 800B of fundamental mode image data 115, 215, 715 described above. FIG. 8A shows an exemplary region C of longitudinal section 800A from which a portion of en face projection 800C can be extracted. Region C of A-line by pullback longitudinal section 800A can be seen to be a region of relatively lower A-line coordinates (when comparted to region B of A-line by pullback longitudinal section 800A) between a lower A-line coordinate threshold 808 and an upper A-line coordinate threshold 806. As discussed above, the A-line by longitudinal section 800A of FIG. 8A represents a single azimuthal value (ϕ) in OCT image data 113, 213, 713. The en face projection 800C of FIG. 8C may be obtained by: for each azimuthal coordinate (ϕ) and for each pullback axis coordinate in OCT image data 113, 213, 713 (i.e. for each azimuthal, pullback coordinate pair in OCT image data 113, 213, 713), summing or averaging values along the A-line direction between lower A-line coordinate threshold 808 and upper A-line coordinate threshold 806 (e.g. in region C shown in FIG. 8A) to obtain a value for an en face pixel corresponding to the current azimuthal, pullback coordinate pair. Since the A-line by pullback longitudinal section 800A shown in FIG. 8A represents only one azimuthal value (ϕ) of OCT image data 113, 213, 713, the data of FIG. 8A only creates one horizontal line of pixels in the FIG. 8C en face image 800C. En face image 800C is a projection in the pullback and azimuthal coordinate (ϕ) axes.

Comparing the exemplary fundamental mode en face projection 800B (FIG. 8B) and exemplary the higher order mode en face projection 800C (FIG. 8C), bright ridges 811 of en face projection 800B correspond to dark creases 821 in en face projection 800C and dark creases 813 in en face projection 800B correspond to bright ridges 823 in en face projection 800C.

En face images (e.g. en face projections 800B, 800C) may be used to generate multipath image data 119, 219, 719 using any suitable techniques. In one exemplary technique, a ratio of fundamental mode and higher order mode en face projections (e.g. en face projections 800B, 800C) may be used to generate multipath image data 119, 210, 710. FIG. 8D shows exemplary multipath image data 119, 219, 719 shown as a multipath en face projection 800D obtained by pixel-wise division between en face projection 800B and en face projection 800C. As can be observed in image 800D, the brighter ridges 831 and darker creases 833 are more strongly emphasized in multipath en face projection 800D by using both fundamental mode image data 115, 215, 715 and higher order mode image data 117, 217, 717 (e.g. by using exemplary en face projections 800B, 800C). The low-angle scattering from ridges 831 result in a relative increase in coupling into the fundamental mode, compared to the higher angled scattering from the creases which correspond more to the higher order modes. The angle at which the sample is imaged impacts the multipath contrast response.

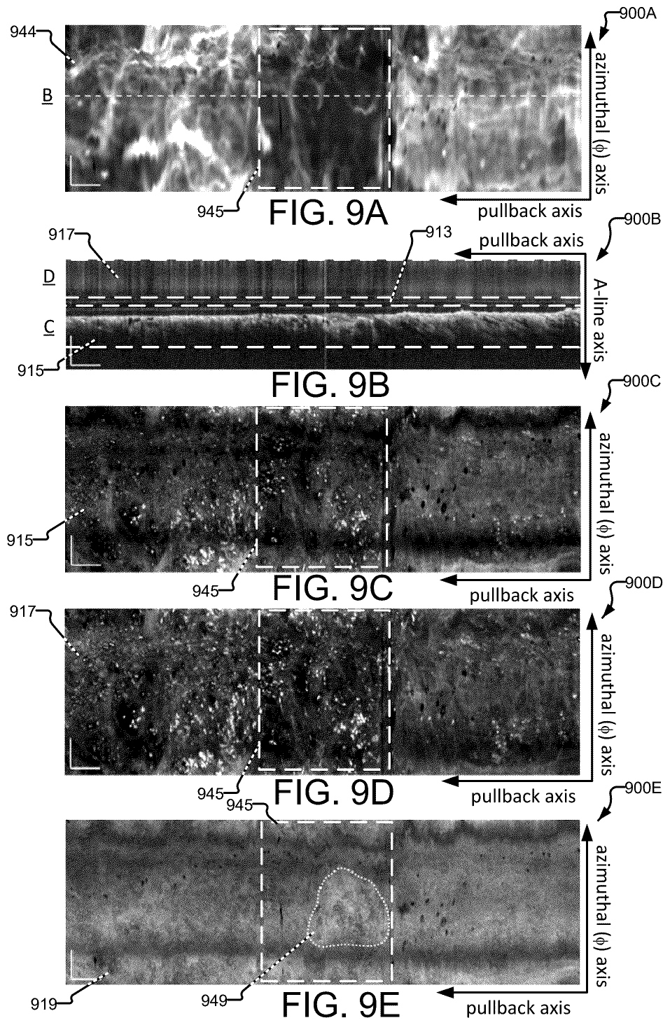

FIGS. 9A-E (collectively, FIG. 9) are depictions of experimental data obtained from imaging a human lung sample exhibiting an adenocarcinoma using a multi-imaging type imaging system of the type disclosed herein (e.g. system 200 comprising an OCT imaging system 202 and a second imaging type system 240 comprising an AFI imaging system 240). The left-hand side of FIGS. 9A-E is a distal end (i.e. deeper into sub-segmental airway) and the right-hand side of FIGS. 9A-E is proximal (i.e. closer to a bronchoscope) along the pullback direction. FIG. 9A depicts AFI image data 944 (i.e. a specific example of second type image data 244), FIG. 9B depicts OCT image data 913 (e.g. similar to OCT image data 113, 213, 713), FIGS. 9C and 9D respectively depict en face projections of fundamental mode image data 915 (e.g. similar to fundamental mode image data 115, 215, 715) and higher order mode image data 917 (e.g. similar to higher order image mode data 117, 217, 717) and FIG. 9D depicts an en face projection of multipath image data 919 (e.g. similar to multipath image data 119, 219, 719).

Pullback by azimuthal section 900A of AFI image data 944 (FIG. 9A) is shown in a plane defined by azimuthal (ϕ) axis and the pullback axis). Pullback by A-line section 900B (FIG. 9B) of OCT image data 913 is shown in a plane defined by an A-line axis (e.g. A-line 411 shown in FIG. 5B) corresponding to one azimuthal (ϕ) coordinate (i.e. one azimuthal value (ϕ) about pullback axis) and a pullback axis (e.g. axis 415 shown in FIG. 5B). Section 900B shown in FIG. 9B represents data from line B in FIG. 9A (and corresponding lines for other azimuthal coordinates (ϕ)). Line B in FIG. 9A bisects darkened autofluorescence region 945 shown in FIG. 9A, therefore visualizing a thickened epithelial layer. FIG. 9C shows an en face projection 900C of fundamental mode image data 915 extracted from OCT image data 913 shown in FIG. 9B (and the additional OCT image data 913 corresponding to other azimuthal (ϕ) coordinates) on the basis of A-line coordinates. FIG. 9B shows an exemplary region C of longitudinal section 900B from which a portion of en face projection 900C can be extracted. FIG. 9D shows an en face projection 900D of higher order mode image data 917 extracted from OCT image data 913 shown in FIG. 9B (and the additional OCT image data 913 corresponding to other azimuthal (ϕ) coordinates) on the basis of A-line coordinates. FIG. 9B also shows an exemplary region D of longitudinal section 900B from which a portion of en face projection 900D of FIG. 9D can be extracted. The en face projections of FIGS. 9C and 9D may be obtained from OCT image data 913 in a manner similar to the en face projections described elsewhere herein with reference to FIG. 8. A ratio of fundamental mode and higher order mode en face projections (e.g. en face projections 900C, 900D) may be used to generate multipath image data 919 shown as a multipath en face projection 900E in FIG. 9E.

In the FIG. 9A AFI image data 1044, it can be observed that a region 945 of AFI image data 944 is noticeably relatively darker than other regions in section AFI image data 944. However, there may be many causes for a darkened region in AFI image data 944. Therefore, it is difficult to draw a definitive diagnostic conclusion about the darkened region 945 on the basis of AFI image data 944 alone. In both en face projections 900C and 900D, region 945 is not easily distinguishable from surrounding regions. However, in multipath image data 919 of FIG. 9E, there is a visible difference between higher contrast region 949 and surrounding regions, suggesting that there is a decrease in backscattering angle over region 949 compared to the surrounding tissue. The higher contrast region 949 within region 945 of multipath image data 919 corroborates the darkened region 945 observed in AFI image data 944.

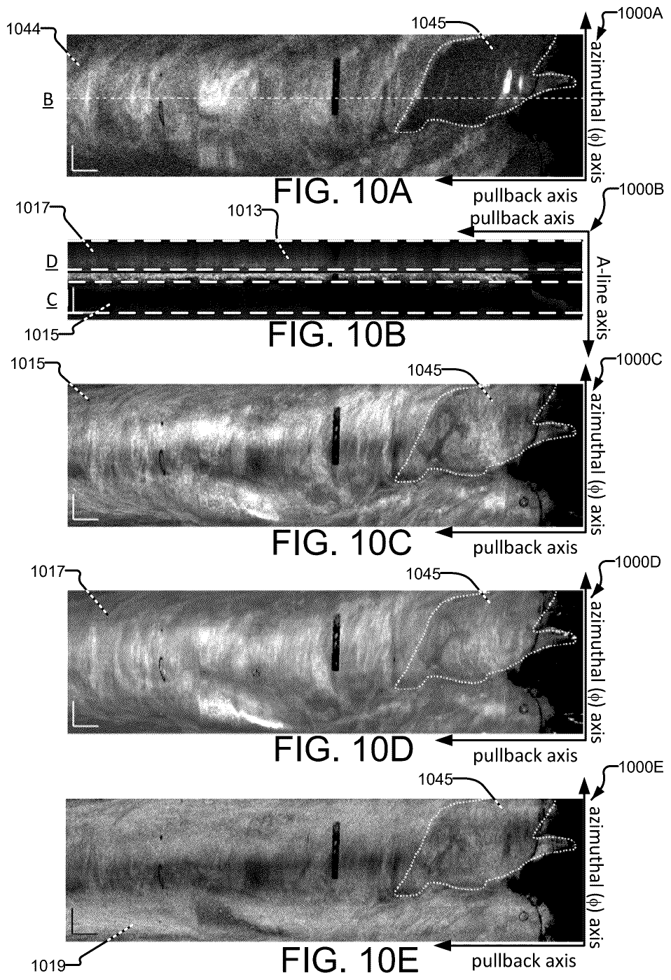

FIGS. 10A-E (collectively, FIG. 10) are depictions of another set of experimental data obtained from ex vivo imaging of a fallopian tube specimen using a multi-imaging type imaging system of the type disclosed herein (e.g. system 200 comprising an OCT imaging system 202 and a second imaging type system 240 comprising an AFI imaging system 240). The left-hand side of FIGS. 10A-E is the isthmus (i.e. uterine end) and the right-hand side of FIGS. 10A-E is the fimbriae (i.e. ovarian) along the pullback direction. FIG. 10A depicts AFI image data 1044 (i.e. a specific example of second type image data 244), FIG. 10B depicts OCT image data 1013 (e.g. similar to OCT image data 113, 213, 713), FIGS. 10C and 10D respectively depict en face projections of fundamental mode image data 1015 (e.g. similar to fundamental mode image data 115, 215, 715) and higher order mode image data 1017 (e.g. similar to higher order image mode data 117, 217, 717) and FIG. 10D depicts an en face projection of multipath image data 1019 (e.g. similar to multipath image data 119, 219, 719).

Pullback by azimuthal section 1000A of AFI image data 1044 (FIG. 10A) is shown in a plane defined by azimuthal (ϕ) axis and the pullback axis). Pullback by A-line section 1000B (FIG. 10B) of OCT image data 1013 is shown in a plane defined by an A-line axis corresponding to one azimuthal (ϕ) coordinate (i.e. one azimuthal value (¢) about pullback axis) and a pullback axis (e.g. pullback axis 415 shown in FIG. 5B). FIG. 10C is an en face projection 1000C of fundamental mode image data 1015 extracted from OCT image data 1013 shown in FIG. 10B (and the additional OCT image data 1013 corresponding to other azimuthal (ϕ) coordinates) on the basis of A-line coordinates. FIG. 10B shows an exemplary region C of longitudinal section 1000B from which a portion of en face projection 1000C can be extracted. FIG. 10D shows an en face projection 1000D of higher order mode image data 1017 extracted from OCT image data 1013 shown in FIG. 10B (and the additional OCT image data 1013 corresponding to other azimuthal (ϕ) coordinates) on the basis of A-line coordinates. FIG. 10B also shows an exemplary region D of longitudinal section 1000B from which a portion of en face projection 1000D of FIG. 10D can be extracted. The en face projections of FIGS. 10C and 10D may be obtained from OCT image data 1013 in a manner similar to the en face projections described elsewhere herein with reference to FIG. 8. A ratio of fundamental mode and higher order mode en face projections (e.g. en face projections 1000C, 1000D) may be used to generate multipath image data 1019 shown as a multipath en face projection 1000E in FIG. 10E.

In the FIG. 10A AFI image data 1044, it can be observed that region 1045 is darkened compared to the surrounding regions of section 1000A. Darkened region 1045 may therefore be a potential indication of high grade serous carcinoma (HGSC). However, it can be seen that such feature is not clearly distinguishable in longitudinal section 1000B of OCT image data 1013, en face projection 1000C of fundamental mode image data 1015 or en face projection 1000D of higher order mode image data 1017 of FIGS. 9B-D respectively. Nevertheless, it can be observed that in multipath image data 1019 shown in en face projection 900E of FIG. 9E, a darkening is observable in region 1045 compared to the surrounding regions of image data 1019. Therefore, multipath image data 1019 may provide corroboration to AFI image data 1044 (e.g. identification of HGSC in fallopian tubes).

In some embodiments, sample 111, 211, 711 is the tissue of a human or animal subject and imaging systems 100, 200, 700 comprise endoscopic imaging systems for use in generating multipath image data 119, 219, 719 that is useful for diagnosis of various medical conditions. This is not necessary. While sample 111, 211, 711 may be tissue of a human or animal subject, sample 111, 211, 711 may comprise other suitable matter for which imaging is desired. While imaging systems 100, 200, 700 may comprise endoscopic imaging systems, imaging systems 100, 200, 700 may comprise fiber optic imaging systems 100, 200, 700 generally. While multipath image data 119, 219, 719 may be useful for diagnosis of various medical conditions, this is not necessary and multipath image data 119, 219, 719 may be used for any suitable purpose.

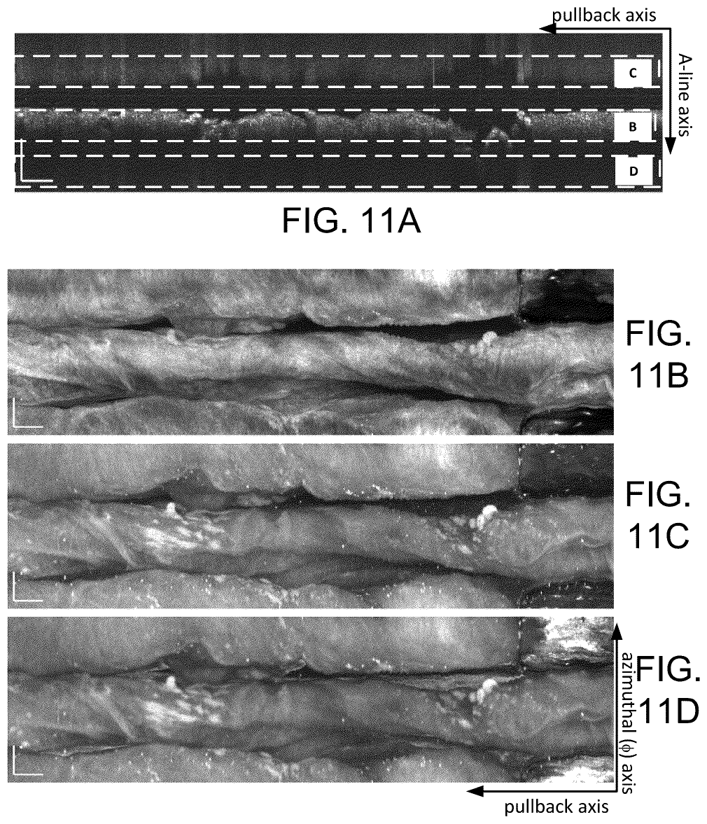

FIGS. 11A-D (collectively, FIG. 11) are depictions of another set experimental data obtained from an ex vivo fallopian tube specimen which shows how multiple components of higher order mode image data (corresponding to higher order mode incident light and fundamental mode incident light) may be extracted from the OCT image data. In the FIG. 11 images of the fallopian tube, the left hand side is the isthmus (uterine end) and the right hand side is the fimbriated (ovarian) end. FIG. 11A shows an A-line by pullback longitudinal section of OCT image data 113, 213, 713 having three regions B, C and D separable by A-line coordinate thresholds. Region B shown in FIG. 11A represents fundamental mode image data 115, 215, 715 that corresponds to image data where sample 111 is illuminated by fundamental mode incident light which is coupled back into the fundamental mode of MCF 212. FIG. 11B is an en face image corresponding to region B in FIG. 11A. Region C shown in FIG. 11A represents higher order mode image data 117, 217, 717 that corresponds to image data where sample 111 is illuminated by fundamental mode incident light which is coupled back into the higher order mode(s) of MCF 212. FIG. 11C is an en face image corresponding to region C in FIG. 11A. Region D shown in FIG. 11A represents additional higher order mode image data 122, 222, 722 that corresponds to image data where sample 111 is illuminated by higher order mode incident light which is coupled back into the higher order mode(s) of MCF 212. FIG. 11C is an en face image corresponding to region C in FIG. 11A. Multipath image data 119 may be based on fundamental mode image data 115, 215, 715 and either or both of higher order mode image data 117, 217, 717 and additional higher order mode image data 122, 222, 722.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, graphics processing units (GPUs), math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

Aspects of the invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, code for configuring a configurable logic circuit, applications, apps, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software and other modules may reside on servers, workstations, personal computers, tablet computers, and other devices suitable for the purposes described herein.

The invention comprises a number of non-limiting aspects. Non-limiting aspects of the invention comprise:

1. A fiber optic imaging system for generating multipath image data, the system comprising:
an optical coherence tomography (OCT) system comprising an OCT fiber optically connected to an interferometric detector;
a multi-clad fiber (MCF) for receiving sampled light that has interacted with a sample and propagating the sampled light as a fundamental MCF mode and one or more higher order MCF modes;
an optical joint for coupling the sampled light from the MCF into the OCT fiber, the optical joint configured to couple at least some light energy from both the fundamental MCF mode and the one or more higher order MCF modes into a fundamental mode of the OCT fiber where the at least some light energy propagates as OCT return light.

2. The imaging system of aspect 1 or any other aspect herein comprising:
a controller connected to the interferometric detector to receive, from the interferometric detector, OCT image data based on the OCT return light, the controller configured to:
   extract fundamental mode image data and higher order mode image data from the OCT image data, wherein the extraction is based on A-line coordinates of the OCT image data; and
   generate multipath image data (e.g. diagnostic image data) based on both of the fundamental mode image data and the higher order mode image data.

3. The imaging system of aspect 2 or any other aspect herein wherein the controller is configured to:
extract the fundamental mode image data based on light energy from the fundamental MCF mode that is coupled into the fundamental mode of the OCT fiber; and
extract the higher order mode image data based on light energy from the one or more higher order MCF modes that is coupled into the fundamental mode of the OCT fiber.

4. The imaging system of any one of aspects 1 to 3 or any other aspect herein wherein the controller is configured to extract the fundamental mode image data and the higher order mode image data from the OCT image data using upper and lower A-line coordinate thresholds.

5. The imaging system of any one of aspects 1 to 4 or any other aspect herein wherein the MCF introduces a phase delay between the fundamental MCF mode and the one or more higher order MCF modes.

6. The imaging system of aspect 5 or any other aspect herein wherein the controller is configured to extract the fundamental mode image data and the higher order mode image data based on the phase delay between the fundamental MCF mode and the one or more higher order MCF modes.

7. The imaging system of any one of aspects 5 to 6 or any other aspect herein wherein an amount of the phase delay depends on a length of the MCF and wherein the amount of the phase delay is sufficient to enable extraction of the fundamental mode image data and the higher order mode image data from the OCT image data based on the A-line coordinates of the OCT image data.

8. The imaging system of any one of aspects 1 to 7 or any other aspect herein wherein the MCF comprises a MCF core and at least one light-transmitting MCF cladding and wherein the fundamental MCF mode propagates substantially in the MCF core and the one or more higher order MCF modes propagate substantially in the at least one MCF cladding.

9. The imaging system of any one of aspects 1 to 8 or any other aspect herein wherein the fundamental MCF mode comprises light energy received from relatively low numerical aperture portions of the sampled light and the one or more higher order MCF modes comprise light energy received from relatively high numerical aperture portions of the sampled light.

10. The imaging system of any one of aspects 1 to 9 or any other aspect herein wherein the MCF propagates incident light from the optical joint toward the sample in an incident direction opposite to a direction of propagation of the sampled light, at least a portion of the incident light interacting with the sample to become the sampled light.

11. The imaging system of aspect 10 or any other aspect herein wherein the incident light comprises: fundamental mode incident light that propagates in the incident direction in a fundamental mode of the MCF; and higher order mode incident light that propagates in the incident direction in one or more higher order modes of the MCF.

12. The imaging system of aspect 11 or any other aspect herein wherein the one or more higher order MCF modes of the sampled light comprise a first component corresponding to the fundamental mode incident light that has interacted with the sample and a second component corresponding to the higher order mode incident light that has interacted with the sample.

13. The imaging system of aspect 12 of any other aspect herein wherein the controller is configured to extract the higher order mode image data based on light energy from the first component of the one or more higher order MCF modes of the sampled light that is coupled into the fundamental mode of the OCT fiber.

14. The imaging system of aspect 13 or any other aspect herein wherein the controller is configured to extract additional higher order mode image data from the OCT image data based on light energy from the second component of the one or more higher order MCF modes of the sampled light that is coupled into the fundamental mode of the OCT fiber.

15. The imaging system of aspect 14 or any other aspect herein wherein the controller is configured to extract the additional higher order mode image data based on A-line coordinates of the OCT image data.

16. The imaging system of any one of aspects 14 to 15 or any other aspect herein wherein the controller is configured to extract the fundamental mode image data, the higher order mode image data and the additional higher order mode image data based on differential phase delay between different propagation modes introduced in the MCF.

17. The imaging system of any one or aspects 14 to 16 or any other aspect herein wherein the controller is configured to generate multipath image data (e.g. diagnostic image data) based at least in part on the additional higher order mode image data.

18. The imaging system of any one of aspects 1 to 17 or any other aspect herein wherein the optical joint is configured to couple at least some light energy from both the fundamental MCF mode and the one or more higher order MCF modes into the fundamental of the OCT fiber by introducing an offset between the MCF and the OCT fiber.

19. The imaging system of aspect 18 or any other aspect herein wherein the offset comprises a spatial offset between an axis of the MCF and an axis of the OCT fiber.

20. The imaging system of any one of aspects 18 to 19 or any other aspect herein wherein the offset comprises an angular offset between an axis of the MCF and an axis of the OCT fiber.

21. The imaging system of any one of aspects 18 to 20 or any other aspect herein wherein the optical joint comprises one or more optical components (e.g. lenses and/or mirrors) which introduce optical offset between the MCF and the OCT fiber.

22. The imaging system of any one of aspects 1 to 21 or any other aspect herein wherein the optical joint comprises a fiber optic rotary joint (FORJ) configured to rotate the MCF about its axis relative to the sample.

23. The imaging system of any one of aspects 1 to 22 or any other aspect herein wherein the fundamental mode image data and the higher order mode image data are volumetric data.

24. The imaging system of any one of aspects 1 to 23 or any other aspect herein wherein the controller is configured to:
process the fundamental mode image data to obtain two-dimensional processed fundamental mode image data;
process the higher order mode image data to obtain two-dimensional processed higher order mode image data; and
generate the multipath image data based on the two-dimensional processed fundamental mode image data and the two-dimensional processed higher order mode image data.

25. The imaging system of aspect 24 or any other aspect herein wherein the controller is configured to:
process the fundamental mode image data to obtain the two-dimensional processed fundamental mode image data as a fundamental mode en face image by repeating: for each for each azimuthal coordinate (ϕ) and for each pullback axis coordinate in the fundamental mode image data, aggregating (e.g. adding or averaging) pixel values along the A-line direction between lower and upper fundamental mode A-line coordinate thresholds to obtain one corresponding pixel value of the two-dimensional processed fundamental mode image data; and
process the higher order mode image data to obtain the two-dimensional processed higher order mode image data as a higher order mode en face image by repeating: for each for each azimuthal coordinate (ϕ) and for each pullback axis coordinate in the higher order mode image data, aggregating (e.g. adding or averaging) pixel values along the A-line direction between lower and upper higher order mode A-line coordinate thresholds to obtain one corresponding pixel value of the two-dimensional processed higher order mode image data.

26. The imaging system of aspect 25 or any other aspect herein wherein the controller is configured to generate the multipath image data based on a combination of the fundamental mode en face image and the higher order mode en face image.

27. The imaging system of any one of aspects 25 to 26 or any other aspect herein wherein the controller is configured to generate the multipath image data based on a ratio of the fundamental mode en face image and the higher order mode en face image.

28. The imaging system of any one of aspects 1 to 27 or any other aspect herein wherein MCF comprises a double-cladded fiber (DCF).

29. The imaging system of any one of aspects 1 to 28 or any other aspect herein comprising a second imaging type system, the second imaging type system configured to direct second incident light from the optical joint toward the sample by propagating the second incident light in the MCF, wherein at least a portion of the second incident light interacts with the sample to become second sampled light and wherein the second imaging type system is configured to collect the second sampled light which propagates back toward the optical joint in the MCF.

30. The imaging system of aspect 29 or any other aspect herein comprising a first light source that generates first incident light, at least a portion of which interacts with the sample to become the sampled light and wherein the second imaging type system comprises a second light source for generating the second incident light.

31. The imaging system of aspect 30 or any other aspect herein wherein a wavelength of the first incident light is different than a wavelength of the second incident light.

32. The imaging system of aspect 3 or any other aspect herein wherein the optical joint comprises a wavelength division multiplexer configured to combine the first incident light and the second incident light to generate combined incident light.

33. The imaging system of aspect 32 or any other aspect herein wherein the optical joint comprises a fiber optic rotary joint (FORJ) configured to rotate the MCF about its axis relative to the sample and for coupling the combined incident light into the MCF.

34. The imaging system of aspect 33 or any other aspect herein wherein the optical joint comprises a multi-clad fiber coupler (MCFC) configured to separate the sampled light from the second sampled light.

35. The imaging system of any one of aspects 29 to 34 wherein the second imaging type system comprises an optical detector configured to detect at least a portion of the second sampled light and wherein the controller is configured to generate a second type image based on the detected portion of the second sampled light.

36. The imaging system of aspect 35 or any other aspect herein wherein the second type image comprises an en face image.

37. The imaging system of any one of aspects 29 to 36 or any other aspect herein wherein the second imaging type system is an autofluorescence imaging (AFI) system.

38. A method for generating multipath image data, the method comprising:
providing a multi-clad fiber (MCF);
receiving sampled light that has interacted with the sample and propagating the sampled light in the MCF as a fundamental MCF mode and one or more higher order MCF modes;
coupling the sampled light from the MCF into an optical coherence tomography (OCT) fiber, wherein coupling the sampled light comprises coupling at least some light energy from both the fundamental MCF mode and the one or more higher order MCF modes into a fundamental mode of the OCT fiber, where the at least some light energy propagates as OCT return light;
generating OCT image data based on the OCT return light;
extracting fundamental mode image data and higher order mode image data from the OCT image data, wherein the extracting is based on A-line coordinates of the OCT image data; and
generating the multipath image data based on both of the fundamental mode image data and the higher order mode image data.

39. The method of aspect 38 or any other aspect herein wherein:
the fundamental mode image data is based on light energy from the fundamental MCF mode that is coupled into the fundamental mode of the OCT fiber; and
the higher order mode image data is based on light energy from the one or more higher order MCF modes that is coupled into the fundamental mode of the OCT fiber.

40. The method of any one of aspects 38 to 39 or any other aspect herein wherein extracting the fundamental mode image data and higher order mode image data from the OCT image data comprises using upper and lower A-line thresholds.

41. The method of any one of aspects 38 to 40 or any other aspect herein wherein the MCF introduces a phase delay between the fundamental MCF mode and the one or more higher order MCF modes.

42. The method of aspect 41 or any other aspect herein wherein extracting the fundamental mode image data and higher order mode image data from the OCT image data comprises is based on the phase delay between the MCF mode and the one or more higher order MCF modes.

43. The method of any one of aspects 41 to 42 or any other aspect herein wherein an amount of the phase delay depends on a length of the MCF and wherein the amount of the phase delay is sufficient to enable extraction of the fundamental mode image data and the higher order mode image data from the OCT image data based on the A-line coordinates of the OCT image data.

44. The method of any one of aspects 38 to 43 or any other aspect herein wherein the MCF comprises a MCF core and at least one light-transmitting MCF cladding and wherein the fundamental MCF mode propagates substantially in the MCF core and the one or more higher order MCF modes propagate substantially in the at least one MCF cladding.

45. The method of any one of aspects 38 to 44 or any other aspect herein wherein the fundamental MCF mode comprises light energy received from relatively low numerical aperture portions of the sampled light and the one or more higher order MCF modes comprise light energy received from relatively high numerical aperture portions of the sampled light.

46. The method of any one of aspects 38 to 45 or any other aspect herein wherein the MCF propagates incident light toward the sample in an incident direction opposite to a direction of propagation of the sampled light, at least a portion of the incident light interacting with the sample to become the sampled light.

47. The method of aspect 46 or any other aspect herein wherein the incident light comprises: fundamental mode incident light that propagates in the incident direction in a fundamental mode of the MCF; and higher order mode incident light that propagates in the incident direction in one or more higher order modes of the MCF.

48. The method of aspect 47 or any other aspect herein wherein the one or more higher order MCF modes of the sampled light comprise a first component corresponding to the fundamental mode incident light that has interacted with the sample and a second component corresponding to the higher order mode incident light that has interacted with the sample.

49. The method of aspect 48 or any other aspect herein wherein extracting the higher order mode image data is based on light energy from the first component of the one or more higher order MCF modes of the sampled light that is coupled into the fundamental mode of the OCT fiber.

50. The method of aspect 49 or any other aspect herein comprising extracting additional higher order mode image data from the OCT image data based on light energy from the second component of the one or more higher order MCF modes of the sampled light that is coupled into the fundamental mode of the OCT fiber.

51. The method of aspect 50 or any other aspect herein comprising extracting the additional higher order mode image data based on A-line coordinates of the OCT image data.

52. The method of any one of aspects 50 to 51 or any other aspect herein comprising extracting the fundamental mode image data, the higher order mode image data and the additional higher order mode image data based on differential phase delay between different propagation modes introduced in the MCF.

53. The method of any one of aspects 50 to 52 or any other aspect herein wherein generating the multipath image data (e.g. diagnostic image data) is based at least in part on the additional higher order mode image data.

54. The method of any one of aspects 38 to 53 or any other aspect herein wherein coupling at least some light energy from both the fundamental MCF mode and the one or more higher order MCF modes into the fundamental of the OCT fiber comprises providing an offset between the MCF and the OCT fiber.

55. The method of aspect 54 or any other aspect herein wherein the offset comprises a spatial offset between an axis of the MCF and an axis of the OCT fiber.

56. The method of any one of aspects 54 to 55 or any other aspect herein wherein the offset comprises an angular offset between an axis of the MCF and an axis of the OCT fiber.

57. The method of any one of aspects 54 to 56 or any other aspect herein wherein providing the offset comprises locating one or more optical components (e.g. lenses and/or mirrors) in an optical path between the MCF and the OCT fiber to thereby introduce optical offset between the MCF and the OCT fiber.

58. The method of any one of aspects 38 to 57 or any other aspect herein wherein coupling at least some light energy from both the fundamental MCF mode and the one or more higher order MCF modes into the fundamental of the OCT fiber comprises locating a fiber optic rotary joint (FORJ) configured to rotate the MCF about its axis relative to the sample in an optical path between the MCF and the OCT fiber.

59. The method of any one of aspects 38 to 58 or any other aspect herein wherein the fundamental mode image data and the higher order mode image data are volumetric data.

60. The method of any one of aspects 38 to 59 or any other aspect herein comprising:
processing the fundamental mode image data to obtain two-dimensional processed fundamental mode image data;
processing the higher order mode image data to obtain two-dimensional processed higher order mode image data; and
generating the multipath image data based on the two-dimensional processed fundamental mode image data and the two-dimensional processed higher order mode image data.

61. The method of aspect 60 or any other aspect herein wherein:
processing the fundamental mode image data to obtain the two-dimensional processed fundamental mode image data comprises processing the fundamental mode image data to obtain a fundamental mode en face image by repeating: for each for each azimuthal coordinate (ϕ) and for each pullback axis coordinate in the fundamental mode image data, aggregating (e.g. adding or averaging) pixel values along the A-line direction between lower and upper fundamental mode A-line coordinate thresholds to obtain one corresponding pixel value of the two-dimensional processed fundamental mode image data; and
processing the higher order mode image data to obtain the two-dimensional processed higher order mode image data comprises processing the higher order mode image data to obtain a higher order mode en face image by repeating: for each for each azimuthal coordinate (ϕ) and for each pullback axis coordinate in the higher order mode image data, aggregating (e.g. adding or averaging) pixel values along the A-line direction between lower and upper higher order mode A-line coordinate thresholds to obtain one corresponding pixel value of the two-dimensional processed higher order mode image data.

62. The method of aspect 61 or any other aspect herein wherein generating the multipath image data comprises generating the multipath image data based on a combination of the fundamental mode en face image and the higher order mode en face image.

63. The method of any one of aspects 61 to 62 or any other aspect herein wherein generating the multipath image data comprises generating the multipath image data based on a ratio of the fundamental mode en face image and the higher order mode en face image.

64. The method of any one of aspects 38 to 63 or any other aspect herein wherein the MCF comprises a double-cladded fiber (DCF).

65. The method of any one of aspects 38 to 64 or any other aspect herein comprising:
providing a second imaging type system;
propagating second incident light toward the sample by propagating the second incident light in the MCF;
wherein at least a portion of the second incident light interacts with the sample to become second sampled light; and
collecting the second sampled light which propagates back toward the second imaging type system in the MCF.

66. The method of aspect 65 or any other aspect herein comprising:
generating first incident light by a first light source, at least a portion of which interacts with the sample to become the sampled light; and
generating the second incident light by a second light source.

67. The method of aspect 66 or any other aspect herein wherein a wavelength of the first incident light is different than a wavelength of the second incident light.

68. The method of aspect 67 or any other aspect herein comprising combining the first incident light and the second incident light to generate combined incident light.

69. The method of aspect 68 or any other aspect herein comprising coupling the combined incident light into the MCF by a fiber optic rotary joint (FORJ).

70. The method of aspect 69 or any other aspect herein comprising separating the sampled light from the second sampled light using a multi-clad fiber coupler (MCFC).

71. The method of any one of aspects 65 to 70 or any other aspect herein comprising:
detecting at least a portion of the second sampled light; and
generating a second type image based on the detected portion of the second sampled light.

72. The method of aspect 71 or any other aspect herein wherein the second type image comprises an en face image.

73. The method of any one of aspects 65 to 72 or any other aspect herein wherein the second imaging type system is an autofluorescence imaging (AFI) system.

74. A fiber optic imaging system for generating multipath image data, the system comprising:
an optical coherence tomography (OCT) system comprising an OCT fiber optically connected to an interferometric detector;
a multi-clad fiber (MCF) for receiving sampled light that has interacted with a sample and propagating the sampled light as a fundamental MCF mode and one or more higher order MCF modes;
an optical joint for coupling the sampled light from the MCF into the OCT fiber, the optical joint providing an offset between the MCF and the OCT fiber to couple at least some light energy from both the fundamental MCF mode and the one or more higher order MCF modes into a fundamental mode of the OCT fiber where the at least some light energy propagates as OCT return light.

75. The imaging system of aspect 40 comprising any of the features, combinations of features and/or sub-combinations of features of any of aspects 1 to 37.

76. Apparatus having any new and inventive feature, combination of features, or sub-combination of features as described herein.

77. Methods having any new and inventive steps, acts, combination of steps and/or acts or sub-combination of steps and/or acts as described herein.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value ±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A fiber optic imaging system for generating multipath image data, the system comprising:
an optical coherence tomography (OCT) system comprising an OCT fiber optically connected to an interferometric detector;
a multi-clad fiber (MCF) for receiving sampled light that has interacted with a sample and propagating the sampled light as a fundamental MCF mode and one or more higher order MCF modes;
an optical joint for coupling the sampled light from the MCF into the OCT fiber, the optical joint configured to couple at least some light energy from both the fundamental MCF mode and the one or more higher order MCF modes into a fundamental mode of the OCT fiber where the at least some light energy propagates as OCT return light.

2. The imaging system of claim 1 comprising:
a controller connected to the interferometric detector to receive, from the interferometric detector, OCT image data based on the OCT return light, the controller configured to:
extract fundamental mode image data and higher order mode image data from the OCT image data, wherein the extraction is based on A-line coordinates of the OCT image data; and
generate multipath image data (e.g. diagnostic image data) based on both of the fundamental mode image data and the higher order mode image data.

3. The imaging system of claim 2 wherein the controller is configured to:
extract the fundamental mode image data based on light energy from the fundamental MCF mode that is coupled into the fundamental mode of the OCT fiber; and
extract the higher order mode image data based on light energy from the one or more higher order MCF modes that is coupled into the fundamental mode of the OCT fiber.

4. The imaging system of claim 1 wherein the controller is configured to extract the fundamental mode image data and the higher order mode image data from the OCT image data using upper and lower A-line coordinate thresholds.

5. The imaging system of claim 1 wherein the MCF introduces a phase delay between the fundamental MCF mode and the one or more higher order MCF modes.

6. The imaging system of claim 5 wherein the controller is configured to extract the fundamental mode image data and the higher order mode image data based on the phase delay between the fundamental MCF mode and the one or more higher order MCF modes.

7. The imaging system of claim 5 wherein an amount of the phase delay depends on a length of the MCF and wherein the amount of the phase delay is sufficient to enable extraction of the fundamental mode image data and the higher order mode image data from the OCT image data based on the A-line coordinates of the OCT image data.

8. The imaging system of claim 1 wherein the MCF comprises a MCF core and at least one light-transmitting MCF cladding and wherein the fundamental MCF mode propagates substantially in the MCF core and the one or more higher order MCF modes propagate substantially in the at least one MCF cladding.

9. The imaging system of claim 1 wherein the fundamental MCF mode comprises light energy received from relatively low numerical aperture portions of the sampled light and the one or more higher order MCF modes comprise light energy received from relatively high numerical aperture portions of the sampled light.

10. The imaging system of claim 1 wherein the MCF propagates incident light from the optical joint toward the sample in an incident direction opposite to a direction of propagation of the sampled light, at least a portion of the incident light interacting with the sample to become the sampled light.

11. The imaging system of claim 10 wherein the incident light comprises: fundamental mode incident light that propagates in the incident direction in a fundamental mode of the MCF; and higher order mode incident light that propagates in the incident direction in one or more higher order modes of the MCF.

12. The imaging system of claim 11 wherein the one or more higher order MCF modes of the sampled light comprise a first component corresponding to the fundamental mode incident light that has interacted with the sample and a second component corresponding to the higher order mode incident light that has interacted with the sample.

13. The imaging system of claim 12 wherein the controller is configured to extract the higher order mode image data based on light energy from the first component of the one or more higher order MCF modes of the sampled light that is coupled into the fundamental mode of the OCT fiber.

14. The imaging system of claim 13 wherein the controller is configured to extract additional higher order mode image data from the OCT image data based on light energy from the second component of the one or more higher order MCF modes of the sampled light that is coupled into the fundamental mode of the OCT fiber.

15. The imaging system of claim 14 wherein the controller is configured to extract the additional higher order mode image data based on A-line coordinates of the OCT image data.

16. The imaging system of claim 14 wherein the controller is configured to extract the fundamental mode image data, the higher order mode image data and the additional higher order mode image data based on differential phase delay between different propagation modes introduced in the MCF.

17. The imaging system of claim 14 wherein the controller is configured to generate multipath image data (e.g. diagnostic image data) based at least in part on the additional higher order mode image data.

18. The imaging system of claim 1 wherein the optical joint is configured to couple at least some light energy from both the fundamental MCF mode and the one or more higher order MCF modes into the fundamental of the OCT fiber by introducing an offset between the MCF and the OCT fiber.

19. The imaging system of claim 18 wherein the offset comprises a spatial offset between an axis of the MCF and an axis of the OCT fiber.

20. A method for generating multipath image data, the method comprising:
providing a multi-clad fiber (MCF);
receiving sampled light that has interacted with the sample and propagating the sampled light in the MCF as a fundamental MCF mode and one or more higher order MCF modes;
coupling the sampled light from the MCF into an optical coherence tomography (OCT) fiber, wherein coupling the sampled light comprises coupling at least some light energy from both the fundamental MCF mode and the one or more higher order MCF modes into a fundamental mode of the OCT fiber, where the at least some light energy propagates as OCT return light;
generating OCT image data based on the OCT return light;
extracting fundamental mode image data and higher order mode image data from the OCT image data, wherein the extracting is based on A-line coordinates of the OCT image data; and
generating the multipath image data based on both of the fundamental mode image data and the higher order mode image data.
